SEQUENCE LISTING (31 sequences)

SEQ ID NO: 1 - DNA, 18 bp, Artificial Sequence
  Description of Artificial Sequence: Synthetic oligonucleotide
ctgatggtttaggactac

SEQ ID NO: 2 - DNA, 18 bp, Artificial Sequence
  Description of Artificial Sequence: Synthetic oligonucleotide
gattggtccactaacgta

SEQ ID NO: 3 - DNA, 18 bp, Artificial Sequence
  Description of Artificial Sequence: Synthetic oligonucleotide
ctattatcatgaggagcg

SEQ ID NO: 4 - skipped

SEQ ID NO: 5 - DNA, 61 bp, Artificial Sequence
  Description of Artificial Sequence: Synthetic probe
aaccatcagtactatcgtaacacatccataagaccatctggcacgattagatgtagtccta

SEQ ID NO: 6 - DNA, 59 bp, Artificial Sequence
  Description of Artificial Sequence: Synthetic probe
ggaccaatcgactcggcgaggagtagatactatggactgtacacatgacttacgttagt

SEQ ID NO: 7 - DNA, 64 bp, Artificial Sequence
  Description of Artificial Sequence: Synthetic probe
tgataatagtcgatacgagtgtataacccaggtgattcgacttgacgcaacgttacgctcctca

SEQ ID NO: 8 - DNA, 15 bp, Artificial Sequence
  Description of Artificial Sequence: Synthetic probe
tacgttagtggacca

SEQ ID NO: 9 - DNA, 12 bp, Artificial Sequence
  Description of Artificial Sequence: Synthetic probe
cggcgaggagta

SEQ ID NO: 10 - DNA, 15 bp, Artificial Sequence
  Description of Artificial Sequence: Synthetic probe
gtagtcctaaaccat

SEQ ID NO: 11 - DNA, 15 bp, Artificial Sequence
  Description of Artificial Sequence: Synthetic probe
atcgtaacacatcca

SEQ ID NO: 12 - DNA, 15 bp, Artificial Sequence
  Description of Artificial Sequence: Synthetic probe
cgctcctcatgataa

SEQ ID NO: 13 - DNA, 15 bp, Artificial Sequence
  Description of Artificial Sequence: Synthetic probe
acgagtgtataaccc

SEQ ID NO: 14 - DNA, 60 bp, Artificial Sequence
  Description of Artificial Sequence: Synthetic probe
aaccatcagagtaaccgggtatctgccgcattatatgaacggtgtctaggggtagtccta

SEQ ID NO: 15 - DNA, 18 bp, Artificial Sequence
  Description of Artificial Sequence: Synthetic oligonucleotide
ctgatggtttaggactac

SEQ ID NO: 16 - DNA, 15 bp, Artificial Sequence
  Description of Artificial Sequence: Synthetic oligonucleotide
tacgttagtggacca

SEQ ID NO: 17 - DNA, 60 bp, Artificial Sequence
  Description of Artificial Sequence: Synthetic probe
ggaccaatctgaaattcattcgcaactctcggcatatcgacggtacctttgtacgttagt

SEQ ID NO: 18 - DNA, 18 bp, Artificial Sequence
  Description of Artificial Sequence: Synthetic oligonucleotide
gattggtccactaacgta

SEQ ID NO: 19 - DNA, 15 bp, Artificial Sequence
  Description of Artificial Sequence: Synthetic oligonucleotide
gtagtcctaaaccat

SEQ ID NO: 20 - DNA, 60 bp, Artificial Sequence
  Description of Artificial Sequence: Synthetic probe
catccacctccagactatatggcccgacacattggcgaactagttgtgcacatcgtaaca

SEQ ID NO: 21 - DNA, 18 bp, Artificial Sequence
  Description of Artificial Sequence: Synthetic oligonucleotide
aggtggatgtgttacgat

SEQ ID NO: 22 - DNA, 15 bp, Artificial Sequence
  Description of Artificial Sequence: Synthetic oligonucleotide
atcgtaacacatcca

SEQ ID NO: 23 - DNA, 60 bp, Artificial Sequence
  Description of Artificial Sequence: Synthetic probe
ccttattctttaactaaatcgccggagaaagcctattggaatgtgcccacgacaatgagc

SEQ ID NO: 24 - DNA, 18 bp, Artificial Sequence
  Description of Artificial Sequence: Synthetic oligonucleotide
agaataagggctcattgt

SEQ ID NO: 25 - DNA, 15 bp, Artificial Sequence
  Description of Artificial Sequence: Synthetic oligonucleotide
acaatgagcccttat

SEQ ID NO: 26 - DNA, 60 bp, Artificial Sequence
  Description of Artificial Sequence: Synthetic probe
taacccttcacacatatcgatttctggaccgcaattaattgacgtgttgccacgagtgta

SEQ ID NO: 27 - DNA, 18 bp, Artificial Sequence
  Description of Artificial Sequence: Synthetic oligonucleotide
gaagggttatacactcgt

SEQ ID NO: 28 - DNA, 15 bp, Artificial Sequence
  Description of Artificial Sequence: Synthetic oligonucleotide
acgagtgtataaccc

SEQ ID NO: 29 - DNA, 60 bp, Artificial Sequence
  Description of Artificial Sequence: Synthetic probe
ccgctttgtactacccggtatgccaaccttaatcgaggtaacgatcctgcaaccgtaaga

SEQ ID NO: 30 - DNA, 18 bp, Artificial Sequence
  Description of Artificial Sequence: Synthetic oligonucleotide
acaaagcggtcttacggt

SEQ ID NO: 31 - DNA, 15 bp, Artificial Sequence
  Description of Artificial Sequence: Synthetic oligonucleotide
accgtaagaccgctt

(19) United States
(12) Patent Application Publication
(10) Pub. No.: US 2021/0381025 A1
(43) Pub. Date: Dec. 9, 2021

(54) METHODS AND KITS FOR DETECTING CELLS USING OLIGONUCLEOTIDE CONJUGATED ANTIBODIES

(71) Applicant: The Board of Trustees of the Leland Stanford Junior University, Stanford, CA (US)

(72) Inventors: Julia Kennedy-Darling, Redwood City, CA (US); Nikolay Samusik, Jacksonville, OR (US)

(21) Appl. No.: 17/285,456

(22) PCT Filed: Oct. 31, 2019
(86) PCT No.: PCT/US2019/059255

§ 371 Date: Apr. 14, 2021

Related U.S. Application Data
(60) Provisional application No. 62/753,854, filed on Oct. 31, 2018.

(51) Int. Cl. C12Q 1/6804

(52) U.S. Cl. CPC C12Q 1/6804

(57) ABSTRACT

The tissue microenvironment is a critical factor to disease mechanism and therapeutic efficiency. Provided here is an imaging platform, methods, and kits which enable measurement of tens of parameters simultaneously within a single tissue, with the potential to reveal unique and important biological associations related to the spatial dimension. The sensitivity of the platform, methods, and kits described herein extends to measure low level markers which can be important for tracking disease progression, diagnosing disease, or both.

### CROSS-REFERENCE

This application claims priority to U.S. Provisional Patent Application No. 62/753,854, filed Oct. 31, 2018, which is entirely incorporated herein by reference.

### BACKGROUND

Biomarker measurements enable the detection of a variety of biological states. For some assays, bulk measurements of a single parameter are sufficient to assess the disease state of a given sample; however, these measurements obscure the single cell resolution data and can obfuscate the underlying heterogeneity of a biologically relevant specimen.

### SUMMARY

Provided herein are methods comprising: contacting a sample comprising a plurality of biological features of interest with a plurality of capture agents, wherein each capture agent is capable of binding to a different biological feature of interest, wherein each capture agent is conjugated to a different oligonucleotide; fixing the capture agents bound to biological features of interest to the sample; contacting each oligonucleotide with a circular nucleic acid primer, wherein a segment of the nucleic acid primer is complimentary to the oligonucleotide, and wherein each oligonucleotide is contacted with a different nucleic acid primer; amplifying the oligonucleotides using the circular nucleic acid primers as a template to yield amplified oligonucleotides; contacting each of a subset of the oligonucleotides with a probe comprising a label to form a probe-amplified oligonucleotide duplex, wherein each probe can bind to only one oligonucleotide; reading the sample to determine the binding pattern for each of the probes, inactivating or removing the labels, and repeating the contacting and reading steps with different probes that bind to a different subset of oligonucleotides.

In some embodiments, the sample is a biological sample. In some embodiments, the sample is selected from the group consisting of a fresh sample, a frozen sample, and a chemically fixed sample. In some embodiments, the sample is a FFPE tissue sample. In some embodiments, the sample comprises a cell. In some embodiments, the sample is selected from the group consisting of a biological tissue, a biological fluid, and a homogenate. In some embodiments, the sample comprises cells. In some embodiments, the cells comprise a rare cell population. In some embodiments, the cells comprise cancer cells. In some embodiments, the cell is selected from the group consisting of an animal cell, a plant cell, a bacterium, a fungal cell, or a protist.

In some embodiments, the sample a human sample or a mouse sample. In some embodiments, the sample comprises a pathogen. In some embodiments, the pathogen is selected from the group consisting of a bacterial cell, a yeast cell, a bacterial cell, a virus, a viral vector, or a prion. In some embodiments, the sample comprises a tumor tissue. In some embodiments, the sample comprises healthy tissue. In some embodiments, the sample is adhered to a slide. In some embodiments, the biological features comprise proteins. In some embodiments, the biological features comprise markers. In some embodiments, at least one of the markers is a low level marker. In some embodiments, the biological features comprise a disease marker. In some embodiments, the biological features comprise a diagnostic marker. In some embodiments, the markers comprise a molecule selected from the group consisting of a transcription factor, a signaling molecule, a diffuse extracellular marker, or a cell surface marker. In some embodiments, the biological features comprise a mutated protein.

In some embodiments, the capture agents comprise an antibody. In some embodiments, the capture agents comprise an antibody fragment. In some embodiments, the antibody fragment is selected from the group consisting of an IgG, an IgM, a polyclonal antibody, a monoclonal antibody, a scFv, a nanobody, a Fab, or a diabody

In some embodiments, each different oligonucleotide is at least 10 nucleotides long. In some embodiments, each different oligonucleotide is at least 25 nucleotides long. In some embodiments, each different oligonucleotide is no more than 100 nucleotides long.

In some embodiments, the fixing comprises crosslinking. In some embodiments, the crosslinking comprises using formaldehyde.

In some embodiments, the circular nucleic acid primer is between 6 nucleotides long and 100 nucleotides long. In some embodiments, the segment of the nucleic acid primer that is complimentary to the oligonucleotide is between 16 nucleotides long and 18 nucleotides long.

In some embodiments, the amplifying is performed using a polymerase. In some embodiments, the polymerase is Phi29 polymerase. In some embodiments, the amplifying step lasts for about 1 hour. In some embodiments, the amplifying step is performed at about 37° C.

In some embodiments, each probe comprises a different label than each other probe. In some embodiments, the probe-amplified oligonucleotide duplex can have a Tₘ of at least 15° C. In some embodiments, the label can be a fluorescent label. In some embodiments, the fluorescent label can be selected from the group consisting of Cy3, Cy5, Alexafluor555, Alexafluor647, Alexafluor750, POPO-3, TOTO-3, POPRO3, and TOPRO3. In some embodiments, the fluorescent label can be attached to the probe by a linker.

In some embodiments, reading the sample comprises fluorescent imaging.

Provided in this disclosure are kits comprising a plurality of antibodies, each conjugated to a unique oligonucleotide; a plurality of primers, each specific to one of the unique oligonucleotides; and a plurality of dyes, each specific to one of the unique oligonucleotides.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a rolling circle amplification scheme wherein signal detection is performed using an extra reporter sequence included in the backbone padlock probe/circular nucleic acid primer, in accordance with some embodiments.

FIG. 2 illustrates a rolling circle amplification scheme wherein signal detection is performed using the same segment of the padlock probe that recognizes the oligonucleotide (“barcode sequence”), in accordance with some embodiments.

FIG. 3 illustrates a computer system that is programmed or otherwise configured to perform or control methods described herein.

FIG. 4 illustrates human fresh frozen tonsil tissue stained with oligonucleotide linked antibodies amplified via RCA and probed with fluorescently labeled probes, in accordance with some embodiments. Panel A shows CD45-BX001 (exposure time-50 ms) and CD4-BX021 (exposure time 20 ms). Panel B shows a zoomed in portion of panel A. Panel C shows CD2-BX002 (exposure time 20 ms). Panel D shows a zoomed in portion of panel C.

FIG. 5 illustrates human fresh frozen paraffin embedded tonsil tissue stained with antibodies linked to oligonucleotides, in accordance with some embodiments. Panels A and B show CD31-CX001 (exposure time-50 ms) and CD3-CX002 (exposure time 20 ms) and. Panel C depicts the tissue sample after removal of the labeled probes via de-hybridization. Panels D and E present zoomed-in regions of panels A and B, respectively.

FIG. 6 illustrates example data collected using a 24 marker antibody panel to stain human tonsil, in accordance with some embodiments.

### DETAILED DESCRIPTION

### Overview

Provided herein are methods, systems, and kits for detecting elements of a sample, which can be applied to measure high parameter data with spatial context. Such measurement can provide mechanistic understanding of key disease states or therapeutic modalities. In some cases, such measurement can enable development of enhanced diagnostic tools.

A plurality of biological features of interest of a sample can be detected by employing a single capture molecule staining step in combination with iterative cycles of applying a label, imaging, and removing the label. In some cases, a capture agent can be fixed to the sample. An amplification step, such as rolling circle amplification (RCA), can be employed between the capture molecule staining step and the iterative cycles, for example to provide an amplification of signal.

Binding capture agents to a sample can allow for the ultimate detection of elements of the sample, e.g., biological features of interest. The capture molecule staining step can comprise contacting a sample comprising a plurality of biological features of interest with a plurality of capture agents, such that each capture agent can be capable of binding a different biological feature of interest. In some cases, each capture agent can be conjugated to a different oligonucleotide.

A fixation step can follow the binding of the capture agents, such that the capture agents can be fixed to the sample. Such a fixation step can allow for the following amplification step to be performed on the tissue surface. In some cases, such a fixation step can allow for reliable multiplexing and/or iterative labeling and imaging steps after amplification.

Oligonucleotides can each be contacted with a circular nucleic acid primer in preparation for an amplification step. In some cases, the circular nucleic acid primer can be non-circular prior to contacting the oligonucleotide, and be circularized once in contact with the oligonucleotide. In some cases, a non-circular nucleic acid can be circularized via ligation once it contacts the oligonucleotide. For example, in some cases, a circular nucleic acid primer can be a template for an RCA reaction. In some cases, a non-circular nucleic acid primer can be a template for RCA after it is circularized to become a circular nucleic acid primer via ligation. Such primers can comprise a segment complimentary to a segment of an oligonucleotide bound to a capture agent. In some cases, such primers can comprise a probe segment. When a probe segment is copied during an amplification step, the copy of the probe segment can be complimentary to a nucleic acid probe. In some cases, the probe segment can have the same sequence as a nucleic acid probe. In some cases, each oligonucleotide can be contacted with a different circular nucleic acid primer.

After applying circular nucleic acids, an RCA reaction can be performed to amplify the sample. The RCA reaction can be performed on the surface of the sample, after capture agents are bound, and in some cases after a crosslinking or fixation step. RCA can provide enhanced sensitivity compared with a similar method performed without such amplification, such as an immunofluorescence method or an immunohistochemistry method. RCA can comprise for example isothermal amplification of circular nucleic acid probes bound to the oligonucleotides, such that the circular nucleic acid probes act as primers for the RCA reaction. In some cases, such as when the circular nucleic acid primer has a probe segment, the RCA reaction can result in the creation of multiple binding sites for labeled probes.

After an RCA reaction, a subset of amplified oligonucleotides can be contacted with a probe comprising a label. These probes can be nucleic acid sequences that can bind to copies of the probe segment created in each RCA reaction. In some cases, these probes can be nucleic acid sequences that can be complimentary to the copies of the probe segment created in each RCA reaction. In some cases, each probe can bind to one of the amplified oligonucleotides in the subset. In some cases, a different probe sequence is used for each different amplified oligonucleotide in the subset. In some cases, each amplified oligonucleotide in the subset can bind to one probe sequence. In this way, each biological feature of interest associated with an oligonucleotide in the subset of amplified oligonucleotides can be associated with a different probe. This can allow for detection of each biological feature of interest.

A sample can be read after hybridizing the probes to determine the binding pattern for each of the probes. This reading can indicate spatial information about the biological feature of interest associated with each of the probes via capture agents. In some cases, reading a sample can comprise detecting a label on one or more of the probes. Reading can be accomplished using any acceptable method appropriate for the detection of the label. For example, if a label is a dye label or a fluorescent label, this can be accomplished by imaging the sample with a white light or fluorescent microscope, respectively. As another example, if a label is an enzyme label, this can be accomplished by providing the enzyme with a substrate, allowing a reaction to occur, and detecting a product of the reaction.

After reading a sample, a label can be inactivated or removed. In some cases, a label can be removed from a probe, while a probe remains on the amplified oligonucleotide. In some cases, a probe with its label can be removed from an amplified oligonucleotide. In some cases, a label can be inactivated, such that a signal can be no longer detected from the probe associated with the label. An inactivation or removal step can allow subsequent rounds of reading the sample to determine the binding pattern for a different set of probes.

Contacting and reading can be repeated with a different set of labeled probes that can bind to a different subset of oligonucleotides. In some cases, a subset of amplified oligonucleotides comprising amplified oligonucleotides not previously probed can be each contacted with a probe comprising a label. In some cases, each amplified oligonucleotide in the new subset can bind a different probe than other amplified oligonucleotides in the new subset. In some cases, each probe can bind to only one amplified oligonucleotide in the new subset.

A reading step can be performed to read the new set of labels to determine the binding pattern for the new set of probes. Thus, reading can indicate spatial information about different biological features of interest than in the first iteration. In some cases, the removal or inactivation of labels, contacting, and reading steps can be repeated. In some cases, the repeating can continue until a predetermined number of or all biological features of interest are detected.

Methods provided herein can provide amplification strategies that can provide increased sensitivity compared with other assays, such as immunohistochemistry or immunofluorescence. These methods can allow multiplexing of an assay, and can enable in some cases the detection of low-level markers or markers for which available antibodies can be relatively weak.

In some methods herein, amplification, the RCA reaction can allow for amplification of signal which can increase the stoichiometry of detection molecules relative to each antibody molecule. This stoichiometry can be increased by at least 5 times, at least 15 times, at least 20 times, at least 25 times, at least 30 times, at least 40 times, at least 50 times, at least 100 times, at least 500 times, at least 1000 times, at least 5000 times, or at least 10000 times compared with other assays.

In some cases, the methods provided herein can allow detection of markers that may be below the detection limit for other detection assays, including some other assays which do not possess an amplification step, as well as some other assays which do possess an amplification step.

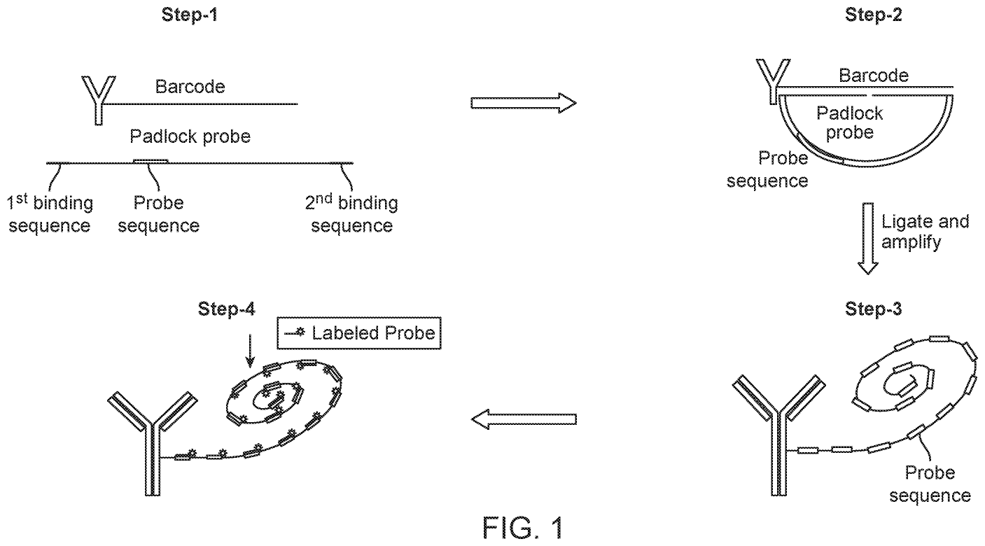

An example of such a method is illustrated in 4 steps in FIG. 1. In the first step, an antibody linked to an oligonucleotide can be bound to a sample (sample not shown). This oligonucleotide can comprise a “barcode,” or a region capable of binding a circular nucleic acid primer. In this illustration, the entire oligonucleotide can serve as the barcode. In other examples, a portion of the oligonucleotide that can be less than the entire oligonucleotide can serve as the barcode. A circular nucleic acid primer (“padlock probe”) can be designed such that one end can be the reverse complement to one portion of the barcode (a first binding sequence) and the other end can be the complement to the other portion of the barcode (a second binding sequence). Between these two ends can be a region having a probe sequence. In the second step, the circular nucleic acid primer can hybridize to the oligonucleotide via the first and second binding sequences, such that the circular nucleic acid primer takes on a circular shape. The ends of the circular nucleic acid primer can be ligated. In the third step, RCA can be performed, extending the oligonucleotide, resulting in an amplified oligonucleotide which can comprise a string of nucleic acids that can be complementary to the padlock probe in a repeating fashion. Notably, the probe sequence can be repeated in this amplified oligonucleotide a plurality of times. In step 4, the amplified oligonucleotide can be incubated with labeled probes that can each comprise a nucleic acid sequence that can be complementary to the probe sequence. Such probes can be linked to a detectable label. The labeled probes can hybridize to the probe sequences on the amplified oligonucleotide, and can be detected, e.g., by imaging. In some cases, this type of method can be called a type 1 RCA method.

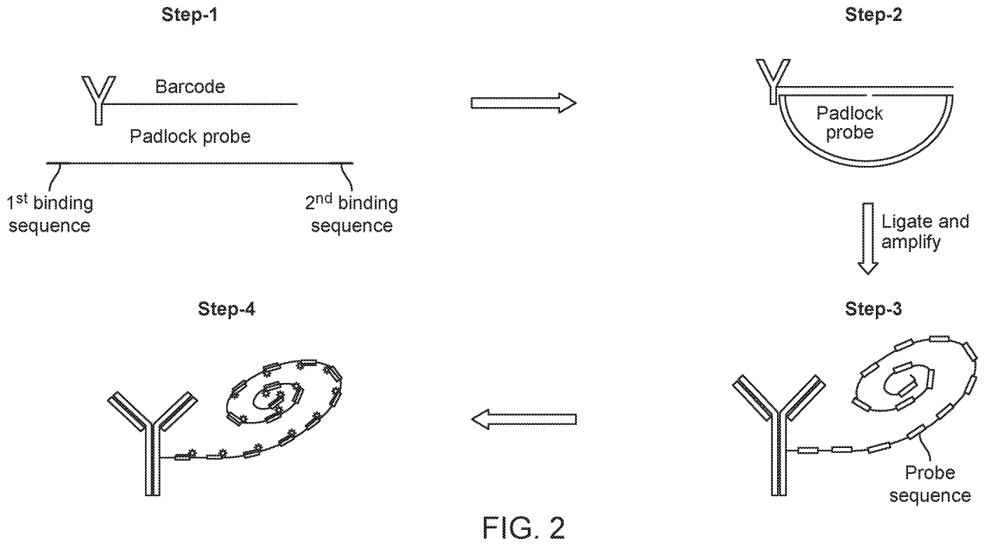

Another example of such a method is illustrated in 4 steps in FIG. 2. In the first step, an antibody linked to an oligonucleotide can be bound to a sample (sample not shown). This oligonucleotide can comprise a “barcode,” or a region that can be capable of binding a circular nucleic acid primer. In this illustration, the entire oligonucleotide can serve as the barcode. In other examples, a portion of the oligonucleotide that can be less than the entire oligonucleotide can serve as the barcode. A circular nucleic acid primer (“padlock probe”) can be designed such that one end can be the reverse complement to one portion of the barcode (a first binding sequence) and the other end can be the complement to the other portion of the barcode (a second binding sequence). These two reporter sequences, when the oligonucleotide is ligated in the next step to a circular shape, can make up a probe sequence. In the second step, the circular nucleic acid primer can hybridize to the oligonucleotide via the first and second binding sequences such that the circular nucleic acid can take on a circular shape. The ends of the circular nucleic acid primer can be ligated. In the third step, RCA can be performed, extending the oligonucleotide, resulting in an amplified oligonucleotide which can comprise a string of nucleic acids that can be complementary to the padlock probe in a repeating fashion. Notably, the probe sequence can be repeated in this amplified oligonucleotide a plurality of times. In step 4, the amplified oligonucleotide can be incubated with labeled probes that can each comprise a nucleic acid sequence that can be complementary to the probe sequence. Such probes can be linked to a detectable label. The labeled probes can hybridize to the amplified oligonucleotide, and can be detected, e.g., by imaging. In some cases, this type of method can be called a type 2 RCA method.

### Samples

A sample can be a biological sample. A sample can be fresh, frozen, or fixed (e.g., chemically fixed). A sample can be of animal, plant, bacteria, fungus, or protist origin. In some cases, a sample can be that of a human, mouse, rat, cow, pig, sheep, monkey, rabbit, fruit fly, frog, nematode or woodchuck. A sample can comprise cells (e.g., isolated cells, immortalized cells, primary cells, cultured cells, or cells of a tissue or organism), biological tissue, biological fluid, a homogenate, or it can be an unknown sample. In some cases, a sample can comprise a pathogen. The pathogen can be cultured or uncultured. A pathogen can be an infection of a sample. In some cases, a pathogen can be an infection of a cell, fluid, tissue, organ, or microbiome of an organism a sample is collected from. In some cases, a sample can comprise a pathogen which is a yeast cell, a bacterial cell, a virus, a viral vector or a prion.

A sample can be a tissue section. In some cases, tissue section can refer to a piece of tissue that has been obtained from a subject, optionally fixed, sectioned, and mounted on a planar surface, e.g., a microscope slide.

A sample can be a planar sample. In some cases, a sample can be immobilized on a surface. In some cases, the surface can be a slide, a plate, a well, a tube, a membrane, a film, or a bead. In some cases, a sample can be contacting a slide. A sample contacting a slide can be attached to the slide such that the sample is effectively immobilized. This can be accomplished for example by fixation or by freezing the sample. Likewise, a sample can be immobilized on another type of surface using a same or similar attachment technique.

In some cases, a sample can be a formalin-fixed paraffin embedded (FFPE) tissue section. FFPE can refer to a piece of tissue, e.g., a biopsy that has been obtained from a subject, fixed, for example in formalin or formaldehyde (e.g., 3%-5% formalin or formaldehyde in phosphate buffered saline) or Bouin solution, embedded in wax, cut into thin sections, and then mounted on a microscope slide.

A sample can be a non-planar sample. A non-planar sample can be a sample that is not substantially flat, e.g., a whole or part organ mount (e.g., of a lymph node, brain, liver, etc.), that has been made transparent by means of a refractive index matching technique such as Clear Lipid-exchanged Acrylamide-hybridized Rigid Imaging-compatible Tissue-hydrogel (CLARITY). See, e.g., Roberts et al., J Vis Exp. 2016; (112): 54025. Clearing agents such as Benzyl-Alcohol/Benzyl Benzoate (BABB) or Benzyl-ether may be used to render a specimen transparent.

The sample may be fixed using an aldehyde, an alcohol, an oxidizing agent, a mercurial, a picrate, or HOPE fixative. In some instances, a sample can be fixed using acetone, formaldehyde, formalin, paraformaldehyde, ethanol, or methanol. The sample may alternatively be fixed using heat fixation. Fixation may be achieved via immersion or perfusion.

In some cases, the biological sample may be frozen. In some cases, the biological sample may be frozen at less than 0° C., less than −10° C., less than −20° C., less than −30° C., less than −40° C., less than −50° C., less than −60° C., less than −70° C., or less than −80° C.

In some cases, a biological sample can be immobilized in a three-dimensional form. Said three-dimensional form can be a frozen block, a paraffin block, or a frozen liquid. For example, a biological sample can be a block of frozen animal tissue in an optimal cutting temperature (OCT) compound. Such a block of tissue can be frozen or fixed. In some cases, a block of tissue can be cut to reveal a surface which can be the surface contacted by the antibody or antibody fragment. Sometimes, a block can be sliced such that serial surfaces of the block can be contacted by the antibody or antibody fragment. In such cases, data which is three-dimensional or approximates three-dimensional data can be acquired.

### Biological Features of Interest

A sample can comprise a biological feature of interest. A biological feature of interest can comprise any part of a sample which can be measured using methods described herein. In some cases, a biological feature of interest can comprise a part of a sample that can be indicated by binding to a capture agent. A biological feature of interest can be a control feature such as a housekeeping feature such as for normalization (e.g., actin), a feature which can identify a part of a cell (e.g., a protein associated with a nucleus, nuclear membrane, endoplasmic reticulum, mitochondria, cell membrane, or other part of the cell), a feature which can identify a type of cell (e.g., a cell surface marker or a protein expressed in a particular cell type, such as an immune cell or a cancer cell), or another feature of interest. In some cases, a biological feature of interest can be a marker of a disease, such as cancer, diabetes, a cardiac disease, a pulmonary disease, an autoimmune disease, an inflammatory disease, or another type of disease. In some cases, a biological feature of interest can be a marker of injury or a marker that is present during would healing. In some cases, a biological feature of interest can be a marker that can indicate a healthy cell. In some cases, a biological feature of interest can be a feature of interest for diagnostic, drug discovery, research, identification, or optimization purposes. In some cases, a biological feature of interest can be an antigen. In some cases, a biological feature of interest can comprise a cell wall, a nucleus, cytoplasm, a membrane, keratin, a muscle fiber, collagen, bone, a protein, a nucleic acid (e.g., mRNA or genomic DNA, etc), fat, etc. A biological feature of interest can also be indicated by immunohistological methods, e.g., using a capture agent that is linked to an oligonucleotide.

A sample can comprise a number of biological features of interest that can be detected using the methods herein. In some cases, the multiplexing features of the method herein (e.g., allowing label to be removed while keeping the capture agents intact on the sample, thus allowing for several or many iterations of the method on a single sample) can be used to detect many biological features of interest. In some cases, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 biological features of interest can be detected. In some cases, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100 biological features of interest can be detected. In some cases, more biological features can be detected in a sample using the present methods than by using other methods, such as non-multiplexed methods, methods wherein a capture agent must be stripped from the sample, methods not including fixing or crosslinking a capture agent to a sample, methods without amplification, or methods with an amplification method different than RCA.

A biological feature of interest can comprise a marker. A marker can be a molecule within a cell, such as a protein, that can inform on the type, disease status, pathogenicity, senescence, or other property of a cell. A marker can in some cases inform a type of cell, such as a lymph cell, a T-cell, a B-cell, a neutrophil, a macrophage, a germ cell, a stem cell, a neural cell, a cancer cell, a healthy cell, an aged cell, an infected cell, or a cell belonging to a particular organ (e.g., a cardiac cell, a Sertoli cell, a hepatocyte, a dermal cell, a thyroid cell, a lung cell, an intestinal cell, a tonsil cell, a muscle cell, a bone cell, a retinal cell such as a rod or a cone, or a cell of another organ). In some cases, a marker can be used to identify a pathogen.

A marker can be a disease marker. A disease marker can be a marker (e.g., a protein) that can be altered in shape, activity, quantity, location, or whether or not it is present or not in a cell having a given disease state. For example, a disease marker can comprise a cancer marker (e.g., a breast cancer marker, a pancreatic cancer marker, a lymphoma marker, a head and neck cancer marker, a gastric cancer marker, a testicular cancer marker, a leukemia marker, a hepatocellular cancer marker, a lung cancer marker, a melanoma marker, an ovarian cancer marker, a thyroid cancer marker, or a marker of another type of cancer), an infectious disease marker (e.g., a marker of a disease caused by a pathogen, such as a marker on the pathogen or a marker of a cell or tissue infected by the pathogen), or a genetic disease marker.

A marker can be a diagnostic marker. A diagnostic marker can be for example a specific biochemical in the body which has a particular molecular feature that makes it useful for detecting a disease, measuring the progress of disease or the effects of treatment, or for measuring a process of interest.

A marker can be a low-level marker, such as a low-level surface marker

### Capture Agents

A capture agent can be a molecule which can bind to a sample. In some cases, a capture agent can bind to a biological feature of interest of a sample. In some cases, a capture agent can specifically bind to a complementary site on a biological feature in a sample. Briefly, a biological feature of interest can be a feature of a sample which can be detected using a capture agent using methods described herein. In some cases, a biological feature of interest can be bound by the capture agent.

A capture agent can be a molecule capable of binding a biological feature. In some cases, a capture agent can comprise a protein, a peptide, an aptamer, or an oligonucleotide. In some cases, a capture agent can comprise an antibody or antigen binding fragment thereof. In some instances, an antibody or an antigen-binding fragment thereof can comprise an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof. It would be understood that antibodies described herein can be modified as known in the art.

A capture agent that is an antibody or antigen binding fragment thereof can comprise a variable region. In some cases, the variable region can comprise a part of an antibody or antigen binding fragment thereof that can contact or specifically bind bind a sample to bind with a biological feature of interest. A variable region can refer to the variable region of an antibody light chain, the variable region of an antibody heavy chain, or a combination of the variable region of an antibody light chain and the variable region of an antibody light chain. In some cases, capture agents which bind different biological features of interest can comprise variable regions which are different in amino acid sequence, protein modifications, three-dimensional structure, or a combination thereof.

A capture agent comprising an antibody or antigen binding fragment thereof can comprise antibody or antibody fragment can comprise an IgG, an IgM, a polyclonal antibody, a monoclonal antibody, a scFv, a nanobody, a Fab, or a diabody. In some cases, an antibody or antigen binding fragment thereof can be of mouse, rat, rabbit, human, camelid, or goat origin. In some cases, an antibody or antigen binding fragment thereof can be raised against a human, mouse, rat, cow, pig, sheep, monkey, rabbit, fruit fly, frog, nematode or woodchuck antigen. In some cases, an antibody or antigen binding fragment thereof can be raised against an animal, plant, bacteria, fungus, or protist antigen. In some cases, the antibody or antigen binding fragment thereof can be raised against a virus, a viral vector, or a prion.

In some cases, the method may comprise labeling the sample with the plurality of capture agents. This step involves contacting the sample (e.g., an FFPE section mounted on a planar surface such as a microscope slide) with all of the capture agents, en masse under conditions by which the capture agents can bind to biological features of interest in the sample. Methods for binding antibodies and aptamers to sites in the sample can be well known.

A capture agent can be in a buffer. In some cases, a capture agent can be applied to a sample in a buffer. A buffer comprising a capture agent can comprise properties which can allow the capture agent to be configured or folded in a state in which the capture agent can bind to a biological feature of interest. In some cases, a buffer comprising a capture agent can comprise properties which can promote binding of the capture agent to a biological feature of interest. In some cases, a buffer comprising a capture agent can comprise properties which can be non-destructive to the capture agent, non-destructive to an oligonucleotide, non-destructive to the sample, or non-destructive to the biological feature of interest.

A capture agent can have specificity for a biological feature of interest. In some cases, a capture agent can have specificity for only one biological feature of interest. In some cases, a capture agent can have specificity for a biological feature of interest that is greater than the specificity of that capture agent for a different biological feature of interest. In some cases, a capture agent can have a specificity for one biological feature of interest that is so much greater than its specificity for other biological features of interest that it can be used to reliably detect the first biological feature of interest.

A capture agent can have affinity for an element of the sample. In some cases, affinity can refer to how fast or how strong the antibody can bind to an element. Affinity can sometimes be described by the dissociation constant (Kd). A capture agent can have a Kd of no more than 10⁻⁴ M, no more than 10⁻⁵M, no more than 10⁻⁶ M, no more than 10⁻⁷ M, no more than 10⁻⁸ M, no more than 10⁻⁹M, no more than 10⁻¹⁰ M, no more than 10⁻¹¹M, no more than 10⁻¹² M, no more than 10⁻¹³ M, or no more than 10⁻¹⁴ M. In some cases, a capture agent can have a Kd of about 10⁻⁴ M, about 10⁻⁵M, about 10⁻⁶ M, about 10⁻⁷ M, about 10⁻⁸ M, about 10⁻⁹M, about 10⁻¹⁰ M, about 10⁻¹¹ M, about 10⁻¹² M, about 10⁻¹³M, or about 10⁻¹⁴ M.

A capture agent can bind to a biological feature of interest at a binding site on a biological feature of interest. Such a binding site, for example, can be an epitope. In some cases, an epitope can be a part of a biological feature of interest. In such a case, the biological feature of interest can comprise an antigen. In some cases, an epitope can bind a capture agent that is an antibody or antigen binding fragment thereof. In such cases, the variable region of the antibody or antigen binding fragment thereof can bind the biological feature of interest at its epitope.

In some cases, a capture agent can be applied to a sample in excess.

In some cases, after a capture agent is contacted with the sample, it can be allowed to incubate for an amount of time. In some cases, a capture agent can be incubated on a sample for at least 30 seconds, at least 1 minute, at least 2 minutes, at least 3 minutes, at least 4 minutes, at least 5 minutes, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 1.5 hours, at least 2 hours, at least 2.5 hours, at least 3 hours, at least 4 hours, at least 5 hours, or at least 6 hours. In some cases, a capture agent can be incubated on a sample for no longer than 30 seconds, no longer than 1 minute, no longer than 2 minutes, no longer than 3 minutes, no longer than 4 minutes, no longer than 5 minutes, no longer than 10 minutes, no longer than 15 minutes, no longer than 20 minutes, no longer than 25 minutes, no longer than 30 minutes, no longer than 35 minutes, no longer than 40 minutes, no longer than 45 minutes, no longer than 50 minutes, no longer than 55 minutes, no longer than 60 minutes, no longer for 1.5 hours, no longer than 2 hours, no longer than 2.5 hours, no longer than 3 hours, no longer than 3.5 hours, no longer than 4 hours, no longer than 4.5 hours, no longer than 5 hours, no longer than 5.5 hours, or no longer than 6 hours. In some cases, a capture agent can be incubated on a sample for between 30 seconds and 6 hours, between 30 seconds and 3 hours, between 30 seconds and 60 minutes, between 30 seconds and 45 minutes, between 30 seconds and 30 minutes, between 30 seconds and 15 minutes, between 30 seconds and 5 minutes, between 30 seconds and 1 minute, between 1 minute and 6 hours, between 1 minute and 3 hours, between 1 minute and 60 minutes, between 1 minute and 45 minutes, between 1 minute and 30 minutes, between 1 minute and 15 minutes, between 1 minute and 5 minutes, between 5 minutes and 6 hours, between 5 minutes and 3 hours, between 5 minutes and 60 minutes, between 5 minutes and 45 minutes, between 5 minutes and 30 minutes, between 5 minutes and 15 minutes, between 15 minutes and 6 hours, between 15 minutes and 3 hours, between 15 minutes and 60 minutes, between 15 minutes and 45 minutes, between 15 minutes and 30 minutes, between 30 minutes and 6 hours, between 30 minutes and 3 hours, between 30 minutes and 60 minutes, between 30 minutes and 45 minutes, between 45 minutes and 6 hours, between 45 minutes and 3 hours, between 45 minutes and 60 minutes, between 60 minutes and 6 hours, or between 60 minutes and 3 hours.

In some cases, after a sample is contacted with a capture agent, the capture agent can be allowed to incubate on the sample at a given temperature. A capture agent can be incubated on the sample at about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., or about 55° C. In some cases, a capture agent can be incubated at a temperature of at least 4° C., at least 5° C., at least 6° C., at least 7° C., at least 8° C., at least 9° C., at least 10° C., at least 11° C., at least 12° C., at least 13° C., at least 14° C., at least 15° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., or at least 55° C. In some cases, a capture agent can be incubated at a temperature of no more than 4° C., no more than 5° C., no more than 6° C., no more than 7° C., no more than 8° C., no more than 9° C., no more than 10° C., no more than 11° C., no more than 12° C., no more than 13° C., no more than 14° C., no more than 15° C., no more than 16° C., no more than 17° C., no more than 18° C., no more than 19° C., no more than 20° C., no more than 21° C., no more than 22° C., no more than 23° C., no more than 24° C., no more than 25° C., no more than 26° C., no more than 27° C., no more than 28° C., no more than 29° C., no more than 30° C., no more than 35° C., no more than 40° C., no more than 45° C., no more than 50° C., or no more than 55° C. In some cases, a capture agent can be incubated at at a temperature between 4° C. and 55° C., between 4° C. and 50° C., between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 55° C., between 10° C. and 50° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 55° C., between 15° C. and 50° C., between 15° C. and 45° C., between 15° C. and 40° C., between 15° C. and 35° C., between 15° C. and 30° C., between 15° C. and 25° C., between 15° C. and 20° C., between 20° C. and 55° C., between 20° C. and 50° C., between 20° C. and 45° C., between 20° C. and 40° C., between 20° C. and 35° C., between 20° C. and 30° C., between 20° C. and 25° C., between 25° C. and 55° C., between 25° C. and 50° C., between 25° C. and 45° C., between 25° C. and 40° C., between 25° C. and 35° C., between 25° C. and 30° C., between 30° C. and 55° C., between 30° C. and 50° C., between 30° C. and 45° C., between 30° C. and 40° C., between 30° C. and 35° C., between 35° C. and 55° C., between 35° C. and 50° C., between 35° C. and 45° C., between 35° C. and 40° C., between 40° C. and 55° C., between 40° C. and 50° C., between 40° C. and 45° C., between 45° C. and 55° C., between 45° C. and 50° C., or between 50° C. and 55° C.

In some cases, after a sample is contacted with a capture agent, excess capture agent can be washed away. In some cases, a wash step can be performed using a wash buffer. A wash buffer can be any buffer than can wash away excess capture agent without significantly impacting the sample, bound capture agent, or oligonucleotide bound to capture agent. In some cases, a wash buffer can comprise PBS, PBS-T, TBS, TBS-T water, saline, or Kreb's buffer.

Excess capture agent can be washed away in one or a plurality of washes. In some cases, about 1, about 2, about 3, about 4, about 5, or about 6 washes can be performed. In some cases, at least 1, at least 2, at least 3, at least 4, at least 5, or at least 6 washes can be performed. In some cases, no more than 1, no more than 2, no more than 3, no more than 4, no more than 5, or no more than 6 washes can be performed. In some cases, between 1 and 6, between 1 and 5, between 1 and 4, between 1 and 3, between 1 and 2, between 2 and 6, between 2 and 5, between 2 and 4, between 2 and 3, between 3 and 6, between 3 and 5, between 3 and 4, between 4 and 6, between 4 and 5, or between 5 and 6 washes can be performed.

Each wash can last about 10 seconds, about 15 seconds, about 30 seconds, about 1 minute, about 2 minutes, about 3 minutes, about 4 minutes, about 5 minutes, about 10 minutes, or about 15 minutes. Each wash can last at least 10 seconds, at least 15 seconds, at least 30 seconds, at least 1 minute, at least 2 minutes, at least 3 minutes, at least 4 minutes, at least 5 minutes, at least 10 minutes, or at least 15 minutes. In some cases, a wash can last for less than 10 seconds. Each wash can last up to 10 seconds, up to 15 seconds, up to 30 seconds, up to 1 minute, up to 2 minutes, up to 3 minutes, up to 4 minutes, up to 5 minutes, up to 10 minutes, or up to 15 minutes. In some cases, a wash can last for more than 15 minutes. Each wash can be between 10 seconds and 15 minutes, between 10 seconds and 10 minutes, between 10 seconds and 5 minutes, between 10 seconds and 1 minute, between 10 seconds and 30 seconds, between 30 seconds and 15 minutes, between 30 seconds and 10 minutes, between 30 seconds and 5 minutes, between 30 seconds and 1 minute, between 1 minute and 15 minutes, between 1 minute and 10 minutes, between 1 minute and 5 minutes, between 5 minutes and 15 minutes, between 5 minutes and 10 minutes, or between 10 minutes and 15 minutes.

Washes can be at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., or about 55° C. In some cases, washes can be at a temperature of at least 4° C., at least 5° C., at least 6° C., at least 7° C., at least 8° C., at least 9° C., at least 10° C., at least 11° C., at least 12° C., at least 13° C., at least 14° C., at least 15° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., or at least 55° C. In some cases, washes can be at a temperature of no more than 4° C., no more than 5° C., no more than 6° C., no more than 7° C., no more than 8° C., no more than 9° C., no more than 10° C., no more than 11° C., no more than 12° C., no more than 13° C., no more than 14° C., no more than 15° C., no more than 16° C., no more than 17° C., no more than 18° C., no more than 19° C., no more than 20° C., no more than 21° C., no more than 22° C., no more than 23° C., no more than 24° C., no more than 25° C., no more than 26° C., no more than 27° C., no more than 28° C., no more than 29° C., no more than 30° C., no more than 35° C., no more than 40° C., no more than 45° C., no more than 50° C., or no more than 55° C. In some cases, washes can be at a temperature between 4° C. and 55° C., between 4° C. and 50° C., between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 55° C., between 10° C. and 50° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 55° C., between 15° C. and 50° C., between 15° C. and 45° C., between 15° C. and 40° C., between 15° C. and 35° C., between 15° C. and 30° C., between 15° C. and 25° C., between 15° C. and 20° C., between 20° C. and 55° C., between 20° C. and 50° C., between 20° C. and 45° C., between 20° C. and 40° C., between 20° C. and 35° C., between 20° C. and 30° C., between 20° C. and 25° C., between 25° C. and 55° C., between 25° C. and 50° C., between 25° C. and 45° C., between 25° C. and 40° C., between 25° C. and 35° C., between 25° C. and 30° C., between 30° C. and 55° C., between 30° C. and 50° C., between 30° C. and 45° C., between 30° C. and 40° C., between 30° C. and 35° C., between 35° C. and 55° C., between 35° C. and 50° C., between 35° C. and 45° C., between 35° C. and 40° C., between 40° C. and 55° C., between 40° C. and 50° C., between 40° C. and 45° C., between 45° C. and 55° C., between 45° C. and 50° C., or between 50° C. and 55° C.

### Oligonucleotides

An oligonucleotide can be a molecule which can be a chain of nucleotides. Oligonucleotides described herein can comprise ribonucleic acids. Oligonucleotides described herein can comprise deoxyribonucleic acids. In some cases, oligonucleotides can be of any sequence, including a user-specified sequence.

Sometimes, an oligonucleotide can comprise G, A, T, U, C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. An oligonucleotide can comprise an LNA, a PNA, a UNA, or an morpholino oligomer, for example. The oligonucleotides used herein may contain natural or non-natural nucleotides or linkages.

An oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, an oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some cases, an oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, an oligonucleotide can be wholly single stranded. In some cases, an oligonucleotide can be partially double stranded. A partially double stranded region can be at the 3′ end of the oligonucleotide, at the 5′ end of the oligonucleotide, or between the 5′ end and 3′ end of the oligonucleotide. In some cases, there may be more than one double stranded region.

In some cases, an oligonucleotide can have a secondary structure. In some cases, an oligonucleotide can have a tertiary structure. Some oligonucleotides can have a structure such that it can fold on itself (e.g. if one region of the oligonucleotide is complementary to another region of the oligonucleotide) to produce one or more double stranded regions comprising a single strand.

In some cases, a segment of an oligonucleotide able to bind a circular nucleic acid primer can be exposed in a single stranded region of the oligonucleotide or an unfolded region of the oligonucleotide. In some cases, a segment of an oligonucleotide able to bind a circular nucleic acid primer can be in a double stranded or folded region of the oligonucleotide, such that upon melting of the oligonucleotide, such a circular nucleic acid primer can bind.

In some cases, an oligonucleotide can be conjugated or bound to a capture agent. In some cases, an oligonucleotide can be conjugated or bound to a capture agent directly using any suitable chemical moiety on the capture agent. In some cases, an oligonucleotide can be linked to a capture agent enzymatically, e.g., by ligation. In some cases, an oligonucleotide can be linked indirectly to a capture agent, for example via a non-covalent interaction such as a biotin/streptavidin interaction or an equivalent thereof, via an aptamer or secondary antibody, or via a protein-protein interaction such as a leucine-zipper tag interaction or the like.

In some cases, an oligonucleotide can be bound to a capture agent using click chemistry, or a similar method. Click chemistry can refer to a class of biocompatible small molecule reactions that can allow the joining of molecules, such as an oligonucleotide and a capture agent. A click reaction can be a one pot reaction, and in some cases is not disturbed by water. A click reaction can generate minimal byproducts, non-harmful byproducts, or no byproducts. A click reaction can be driven by a large thermodynamic force. In some cases, a click reaction can be driven quickly and/or irreversibly to a high yield of a single reaction product (e.g., oligonucleotide conjugated to capture agent), and can have high reaction specificity. Click reactions can include but are not limited to [3+2] cycloadditions, thiol-ene reactions, Diels-Alder reactions, inverse electron demand Diels-Alder reactions, [4+1] cycloadditions, nucleophilic substitutions, carbonyl-chemistry-like formation of ureas, or addition reactions to carbon-carbon double bonds (e.g., dihydroxylation).

In some cases, one or more segments of such an oligonucleotide can be complimentary to one or more segments of nucleic acids of a circular nucleic acid primer. Such a segment can be between 3 and 30 nucleic acids long, between 3 and 20 nucleic acids long, between 3 and 10 nucleic acids long, between 5 and 30 nucleic acids long, between 5 and 20 nucleic acids long, between 5 and 10 nucleic acids long, between 10 and 30 nucleic acids long, between 10 and 20 nucleic acids long, or between 20 and 30 nucleic acids long. In some cases, such a segment can be at least 3 nucleic acids long, at least 5 nucleic acids long, at least 10 nucleic acids long, at least 15 nucleic acids long, at least 20 nucleic acids long, at least 25 nucleic acids long, or at least 30 nucleic acids long. In some cases, such a segment can be no more than 3 nucleic acids long, no more than 5 nucleic acids long, no more than 10 nucleic acids long, no more than 15 nucleic acids long, no more than 20 nucleic acids long, no more than 25 nucleic acids long, or no more than 30 nucleic acids long.

### Fixation and Crosslinking.

Capture agents can be fixed to the sample. In some cases, only capture agents which are bound to a biological feature of interest can be fixed to a sample. In some cases, all capture agents which are bound to a biological feature of interest can be fixed to a sample. Fixation of capture agents to a sample can be performed in some cases after excess (e.g., unbound) capture agent is washed away.

In some embodiments, the capture agents may be cross-linked or fixed to the sample, thereby preventing the capture agent from disassociating during subsequent steps. In some cases, cross-linking can prevent a capture agent from dissociating during an RCA reaction or during inactivation or removal of one or more labels. Thus, fixation or cross-linking of the capture agent to the sample can allow for an RCA reaction to be performed on the sample (rather than in solution), and can allow for multiplexing of the assay by permitting multiple iterations of reading to be performed, as the labels can be removed or inactivated without disturbing the capture agents. This crosslinking step may be done using any amine-to-amine crosslinker (e.g. formaldehyde, disuccinimiyllutarate or another reagent of similar action) although a variety of other chemistries can be used to cross-link the capture agent to the sample if desired.

### Circular Nucleic Acid Primers

A circular nucleic acid primer herein can be a nucleic acid molecule that can be used as a template for an RCA reaction. In some cases, an oligonucleotide can be contacted with a circular nucleic acid primer, and an RCA reaction can be used to lengthen the oligonucleotide according to the sequence of the circular nucleic acid primer.

A circular nucleic acid primer can be a molecule which can be a chain of nucleotides, which can be circular. In some cases, a circular nucleic acid primer does not have an end, such that if polymerase chain reaction amplification were performed using the circular nucleic acid primer, the amplification would not be limited by the length of the primer.

A nucleic acid primer can be a molecule which can be a chain of nucleotides that is circular. Circular can mean that the 3′ end of every nucleic acid in the chain can be connected to the 5′ end of another nucleic acid in the chain, and the 5′ end of every nucleic acid in the chain can be connected to the 3′ end of another nucleic acid in the chain. Circular nucleic acid primers described herein can comprise ribonucleic acids. Circular nucleic acid primers described herein can comprise deoxyribonucleic acids. In some cases, a circular nucleic acid primer can be of any sequence, including a user-specified sequence.

Sometimes, a circular nucleic acid primer can comprise G, A, T, U, C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known.

A circular nucleic acid primer can comprise a segment which can be complimentary to one or more segments of an oligonucleotide. Such a segment can be between 3 and 30 nucleic acids long, between 3 and 20 nucleic acids long, between 3 and 10 nucleic acids long, between 5 and 30 nucleic acids long, between 5 and 20 nucleic acids long, between 5 and 10 nucleic acids long, between 10 and 30 nucleic acids long, between 10 and 20 nucleic acids long, or between 20 and 30 nucleic acids long. In some cases, such a segment can be at least 3 nucleic acids long, at least 5 nucleic acids long, at least 10 nucleic acids long, at least 15 nucleic acids long, at least 20 nucleic acids long, at least 25 nucleic acids long, or at least 30 nucleic acids long. In some cases, such a segment can be no more than 3 nucleic acids long, no more than 5 nucleic acids long, no more than 10 nucleic acids long, no more than 15 nucleic acids long, no more than 20 nucleic acids long, no more than 25 nucleic acids long, or no more than 30 nucleic acids long. In some instances, such a segment can be 15 nucleic acids long, 16 nucleic acids long, 17 nucleic acids long, 18 nucleic acids long, 19 nucleic acids long, or 20 nucleic acids long. Such a segment can be between 15 nucleic acids long and 20 nucleic acids long, between 15 nucleic acids long and 19 nucleic acids long, between 15 nucleic acids long and 18 nucleic acids long, between 15 nucleic acids long and 17 nucleic acids long, between 15 nucleic acids long and 16 nucleic acids long, between 16 nucleic acids long and 20 nucleic acids long, between 16 nucleic acids long and 19 nucleic acids long, between 16 nucleic acids long and 18 nucleic acids long, between 16 nucleic acids long and 17 nucleic acids long, between 17 nucleic acids long and 20 nucleic acids long, between 17 nucleic acids long and 19 nucleic acids long, between 17 nucleic acids long and 18 nucleic acids long, between 18 nucleic acids long and 20 nucleic acids long, between 18 nucleic acids long and 19 nucleic acids long, or between 19 nucleic acids long and 20 nucleic acids long.

A circular nucleic acid primer can comprise a probe segment. A probe segment can be a segment which when copied, the copy can be complimentary to a nucleic acid probe. A probe segment can be between 3 and 30 nucleic acids long, between 3 and 20 nucleic acids long, between 3 and 10 nucleic acids long, between 5 and 30 nucleic acids long, between 5 and 20 nucleic acids long, between 5 and 10 nucleic acids long, between 10 and 30 nucleic acids long, between 10 and 20 nucleic acids long, or between 20 and 30 nucleic acids long. In some cases, a probe segment can be at least 3 nucleic acids long, at least 5 nucleic acids long, at least 10 nucleic acids long, at least 15 nucleic acids long, at least 20 nucleic acids long, at least 25 nucleic acids long, or at least 30 nucleic acids long. In some cases, a probe segment can be no more than 3 nucleic acids long, no more than 5 nucleic acids long, no more than 10 nucleic acids long, no more than 15 nucleic acids long, no more than 20 nucleic acids long, no more than 25 nucleic acids long, or no more than 30 nucleic acids long. In some instances, such a segment can be 15 nucleic acids long, 16 nucleic acids long, 17 nucleic acids long, 18 nucleic acids long, 19 nucleic acids long, or 20 nucleic acids long. Such a segment can be between 15 nucleic acids long and 20 nucleic acids long, between 15 nucleic acids long and 19 nucleic acids long, between 15 nucleic acids long and 18 nucleic acids long, between 15 nucleic acids long and 17 nucleic acids long, between 15 nucleic acids long and 16 nucleic acids long, between 16 nucleic acids long and 20 nucleic acids long, between 16 nucleic acids long and 19 nucleic acids long, between 16 nucleic acids long and 18 nucleic acids long, between 16 nucleic acids long and 17 nucleic acids long, between 17 nucleic acids long and 20 nucleic acids long, between 17 nucleic acids long and 19 nucleic acids long, between 17 nucleic acids long and 18 nucleic acids long, between 18 nucleic acids long and 20 nucleic acids long, between 18 nucleic acids long and 19 nucleic acids long, or between 19 nucleic acids long and 20 nucleic acids long.

In some cases, a circular nucleic acid primer can comprise both a probe segment and a segment complementary to one or more segments of an oligonucleotide. In some cases, such a circular nucleic acid primer can be about 30 nucleic acids long, about 40 nucleic acids long, about 50 nucleic acids long, about 60 nucleic acids long, about 70 nucleic acids long, about 80 nucleic acids long, about 90 nucleic acids long, about 100 nucleic acids long, about 110 nucleic acids long, or about 120 nucleic acids long. In some cases, such a circular nucleic acid primer can be at least 30 nucleic acids long, at least 40 nucleic acids long, at least 50 nucleic acids long, at least 60 nucleic acids long, at least 70 nucleic acids long, at least 80 nucleic acids long, at least 90 nucleic acids long, at least 100 nucleic acids long, at least 110 nucleic acids long, or at least 120 nucleic acids long. In some cases, such a circular nucleic acids primer can be not more than 30 nucleic acids long, not more than 40 nucleic acids long, not more than 50 nucleic acids long, not more than 60 nucleic acids long, not more than 70 nucleic acids long, not more than 80 nucleic acids long, not more than 90 nucleic acids long, not more than 100 nucleic acids long, not more than 110 nucleic acids long, or not more than 120 nucleic acids long. In some cases, such a circular nucleic acid primer can be between 30 nucleic acids long and 120 nucleic acids long, between 30 nucleic acids long and 110 nucleic acids long, between 30 nucleic acids long and 100 nucleic acids long, between 30 nucleic acids long and 90 nucleic acids long, between 30 nucleic acids long and 80 nucleic acids long, between 30 nucleic acids long and 70 nucleic acids long, between 30 nucleic acids long and 60 nucleic acids long, between 30 nucleic acids long and 50 nucleic acids long, between 30 nucleic acids long and 40 nucleic acids long, between 40 nucleic acids long and 120 nucleic acids long, between 40 nucleic acids long and 110 nucleic acids long, between 40 nucleic acids long and 100 nucleic acids long, between 40 nucleic acids long and 90 nucleic acids long, between 40 nucleic acids long and 80 nucleic acids long, between 40 nucleic acids long and 70 nucleic acids long, between 40 nucleic acids long and 60 nucleic acids long, between 40 nucleic acids long and 50 nucleic acids long, between 50 nucleic acids long and 120 nucleic acids long, between 50 nucleic acids long and 110 nucleic acids long, between 50 nucleic acids long and 100 nucleic acids long, between 50 nucleic acids long and 90 nucleic acids long, between 50 nucleic acids long and 80 nucleic acids long, between 50 nucleic acids long and 70 nucleic acids long, between 50 nucleic acids long and 60 nucleic acids long, between 60 nucleic acids long and 120 nucleic acids long, between 60 nucleic acids long and 110 nucleic acids long, between 60 nucleic acids long and 100 nucleic acids long, between 60 nucleic acids long and 90 nucleic acids long, between 60 nucleic acids long and 80 nucleic acids long, between 60 nucleic acids long and 70 nucleic acids long, between 70 nucleic acids long and 120 nucleic acids long, between 70 nucleic acids long and 110 nucleic acids long, between 70 nucleic acids long and 100 nucleic acids long, between 70 nucleic acids long and 90 nucleic acids long, between 70 nucleic acids long and 80 nucleic acids long, between 80 nucleic acids long and 120 nucleic acids long, between 80 nucleic acids long and 110 nucleic acids long, between 80 nucleic acids long and 100 nucleic acids long, between 80 nucleic acids long and 90 nucleic acids long, between 90 nucleic acids long and 120 nucleic acids long, between 90 nucleic acids long and 110 nucleic acids long, between 90 nucleic acids long and 100 nucleic acids long, between 100 nucleic acids long and 120 nucleic acids long, between 100 nucleic acids long and 110 nucleic acids long, or between 110 nucleic acids long and 120 nucleic acids long.

In some cases, a circular nucleic acid primer can be a padlock primer. In some cases, a padlock primer can be a linear oligonucleotide that, upon contact with the oligonucleotide linked to the capture agent, can be then fused to form a circular oligonucleotide.

The amino acids at the 5′ end of a padlock probe can be designed to be complimentary to the reverse complement of a portion of an oligonucleotide, while the amino acids at the 3′ end of a padlock probe can be designed to be complimentary to the complement of a portion of the 3′ end of the oligonucleotide. In this way, when the padlock probe contacts the oligonucleotide, hybridization of base pairs can occur such that the padlock probe bound to the barcode sequence in a circular fashion.

A circular nucleic acid probe can be circular prior to contacting an oligonucleotide.

A circular nucleic acid probe can be a linear nucleic acid primer that upon contact with the oligonucleotide takes on a circular shape. In such cases, the primer can be complementary to an oligonucleotide at the 5′ end and the 3′ end of the circular nucleic acid probe. In some cases, at least 3, at least 4, at least 5, at least 6, at least 7, at least 8, at least 9, at least 10, at least 11, or at least 12 nucleic acids on the 5′ end of the primer can be complimentary to nucleic acids on an oligonucleotide. In some cases, at least 3, at least 4, at least 5, at least 6, at least 7, at least 8, at least 9, at least 10, at least 11, or at least 12 nucleic acids on the 3′ end of the primer can be complimentary to nucleic acids on an oligonucleotide. In some cases, no more than 3, no more than 4, no more than 5, no more than 6, no more than 7, no more than 8, no more than 9, no more than 10, no more than 11, or no more than 12 nucleic acids on the 5′ end of the primer can be complimentary to nucleic acids on an oligonucleotide. In some cases, no more than 3, no more than 4, no more than 5, no more than 6, no more than 7, no more than 8, no more than 9, no more than 10, no more than 11, or no more than 12 nucleic acids on the 3′ end of the primer can be complimentary to nucleic acids on an oligonucleotide. In some cases, between 3 and 12, between 3 and 11, between 3 and 10, between 3 and 9, between 3 and 8, between 3 and 7, between 3 and 6, between 3 and 5, between 3 and 4, between 4 and 12, between 4 and 11, between 4 and 10, between 4 and 9, between 4 and 8, between 4 and 7, between 4 and 6, between 4 and 5, between 5 and 12, between 5 and 11, between 5 and 10, between 5 and 9, between 5 and 8, between 5 and 7, between 5 and 6, between 6 and 12, between 6 and 11, between 6 and 10, between 6 and 9, between 6 and 8, between 6 and 7, between 7 and 12, between 7 and 11, between 7 and 10, between 7 and 9, between 7 and 8, between 8 and 12, between 8 and 11, between 8 and 10, between 8 and 9, between 9 and 12, between 9 and 11, between 9 and 10, between 10 and 12, between 10 and 11, or between 11 and 12 nucleic acids on the 5′ end of the primer can be complimentary to nucleic acids on the oligonucleotide. In some cases, between 3 and 12, between 3 and 11, between 3 and 10, between 3 and 9, between 3 and 8, between 3 and 7, between 3 and 6, between 3 and 5, between 3 and 4, between 4 and 12, between 4 and 11, between 4 and 10, between 4 and 9, between 4 and 8, between 4 and 7, between 4 and 6, between 4 and 5, between 5 and 12, between 5 and 11, between 5 and 10, between 5 and 9, between 5 and 8, between 5 and 7, between 5 and 6, between 6 and 12, between 6 and 11, between 6 and 10, between 6 and 9, between 6 and 8, between 6 and 7, between 7 and 12, between 7 and 11, between 7 and 10, between 7 and 9, between 7 and 8, between 8 and 12, between 8 and 11, between 8 and 10, between 8 and 9, between 9 and 12, between 9 and 11, between 9 and 10, between 10 and 12, between 10 and 11, or between 11 and 12 nucleic acids on the 3′ end of the primer can be complimentary to nucleic acids on an oligonucleotide.

Oligonucleotides and circular nucleic acid primers can be synthesized using established oligonucleotide synthesis methods to afford any desired sequence of nucleotides. Methods of synthesizing oligonucleotides are well known in the art. Such methods can range from standard enzymatic digestion followed by nucleotide fragment isolation (see for example, Sambrook, et al., Molecular Cloning: A Laboratory Manual, Third Edition, Cold Spring Harbor, N.Y., (2000), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### Amplification

Amplification can comprise a mechanism for increasing the number of labels that can become associated with a biological feature of interest in a sample. In some methods herein, amplification can comprise a mechanism for increasing the number of labeled probes that can become associated with a biological feature of interest in a sample. In some cases, amplification can be accomplished using a mechanism or method to increase the number of places on an oligonucleotide that a labeled probe can bind, thus increasing the number of labeled probes that can become associated with a biological feature of interest.

Amplification can occur after the fixation step. In some cases, the fixation step can allow the capture agent to be fixed to the sample. In such cases, an oligonucleotide linked to the capture agent can be indirectly linked to the sample. In some cases, this oligonucleotide can be amplified.

An amplification reaction can comprise a reaction which can produce one or more copies of a DNA template. In some cases, the DNA template can be a circular nucleic acid primer. The DNA template can comprise a region which can be complimentary to a region of the oligonucleotide conjugated to the capture agent.

Amplification can comprise an RCA reaction. In an RCA reaction, a circular nucleic acid primer can be used as a template for extension of an oligonucleotide. In some such cases, the amplification can comprise producing one or more copies of the circular nucleic acid primer, which can be connected to an oligonucleotide which is connected to a capture agent. Herein, the oligonucleotide to be extended can be an oligonucleotide conjugated to a capture molecule.

Components for carrying out an RCA reaction can be added to the sample. In some cases, components can be added to the sample after a fixation or crosslinking step. Components can be applied to the sample before a circular nucleic acid primer is applied, at the same time a circular nucleic acid primer is applied, or after a circular nucleic acid primer is applied. Components can be applied individually or as a mixture of components.

Components can comprise a polymerase. A polymerase can be a DNA polymerase (e.g., to amplify a DNA sequence) or an RNA polymerase (e.g., to amplify an RNA sequence). Examples of DNA polymerases can include but are not limited to Phi29, Bst, or Vent. Examples of RNA polymerases can include but are not limited to T7 RNA polymerase.

Components can comprise a suitable buffer, such as a buffer that is compatible with the polymerase. In some cases, a buffer for amplification can comprise Tris-HCl. A buffer can comprise about 10 mM, Tris-HCl, about 15 mM Tris-HCl, about 20 mM Tris-HCl, about 25 mM Tris-HCl, or about 30 mM Tris-HCl.

In some cases, a buffer for amplification can comprise magnesium chloride (MgCl₂). A buffer can comprise at least 1 mM MgCl₂, at least 1.5 mM MgCl₂, at least 2.0 mM MgCl₂, at least 2.5 mM MgCl₂, at least 3.0 mM MgCl₂, at least 3.5 mM MgCl₂, or at least 4.0 mM MgCl₂.

A buffer for amplification can comprise not more than 1 mM MgCl₂, not more than 1.5 mM MgCl₂, not more than 2.0 mM MgCl₂, not more than 2.5 mM MgCl₂, not more than 3.0 mM MgCl₂, not more than 3.5 mM MgCl₂, or not more than 4.0 mM MgCl₂.

A buffer for amplification can comprise between 1.0 mM MgCl₂ and 4.0 mM MgCl₂, between 1.0 mM MgCl₂ and 3.5 mM MgCl₂, between 1.0 mM MgCl₂ and 3.0 mM MgCl₂, between 1.0 mM MgCl₂ and 2.5 mM MgCl₂, between 1.0 mM MgCl₂ and 2.0 mM MgCl₂, between 1.0 mM MgCl₂ and 1.5 mM MgCl₂, between 1.5 mM MgCl₂ and 4.0 mM MgCl₂, between 1.5 mM MgCl₂ and 3.5 mM MgCl₂ between 1.5 mM MgCl₂ and 3.0 mM MgCl₂, between 1.5 mM MgCl₂ and 2.5 mM MgCl₂, between 1.5 mM MgCl₂ and 2.0 mM MgCl₂, between 2.0 mM MgCl₂ and 4.0 mM MgCl₂, between 2.0 mM MgCl₂ and 3.5 mM MgCl₂, between 2.0 mM MgCl₂ and 3.0 mM MgCl₂, between 2.0 mM MgCl₂ and 2.5 mM MgCl₂, between 2.5 mM MgCl₂ and 4.0 mM MgCl₂, between 2.5 mM MgCl₂ and 3.5 mM MgCl₂, between 2.5 mM MgCl₂ and 3.0 mM MgCl₂, between 3.0 mM MgCl₂ and 4.0 mM MgCl₂, between 3.0 mM MgCl₂ and 4.5 mM MgCl₂, or between 3.5 mM MgCl₂ and 4.0 mM MgCl₂.

A buffer for amplification can comprise potassium chloride (KCl). A buffer can comprise at least 1 mM KCl, at least 5 mM KCl, at least 10 mM KCl, at least 15 mM KCl, or at least 20 mM KCl.

A buffer for amplification can comprise not more than 1 mM KCl, not more than 5 mM KCl, not more than 10 mM KCl, not more than 15 mM KCl, or not more than 20 mM KCl.

A buffer for amplification can comprise about 1 mM KCl, about 5 mM KCl, about 10 mM KCl, about 15 mM KCl, or about 20 mM KCl.

A buffer for amplification can comprise between 1 mM KCl and 20 mM KCl, between 1 mM KCl and 15 mM KCl, between 1 mM KCl and 10 mM KCl, between 1 mM KCl and 5 mM KCl, between 5 mM KCl and 20 mM KCl, between 5 mM KCl and 15 mM KCl, between 5 mM KCl and 10 mM KCl, between 10 mM KCl and 20 mM KCl, between 10 mM KCl and 15 mM KCl, or between 15 mM KCl and 20 mM KCl.

In some cases, a buffer for amplification can comprise DMSO. A buffer can comprise at least 0.5% v/v DMSO, at least 1% v/v DMSO, at least 2% v/v DMSO, at least 3% v/v DMSO, at least 4% v/v DMSO, at least 5% v/v DMSO, at least 6% v/v DMSO, at least 7% v/v DMSO, at least 8% v/v DMSO, at least 9% v/v DMSO, at least 10% v/v DMSO, at least 15% v/v DMSO, at least 20% v/v DMSO, at least 25% v/v DMSO, or at least 30% v/v DMSO.

A buffer for amplification can comprise not more than 0.5% v/v DMSO, not more than 1% v/v DMSO, not more than 2% v/v DMSO, not more than 3% v/v DMSO, not more than 4% v/v DMSO, not more than 5% v/v DMSO, not more than 6% v/v DMSO, not more than 7% v/v DMSO, not more than 8% v/v DMSO, not more than 9% v/v DMSO, or not more than 10% v/v DMSO, not more than 15% v/v DMSO, not more than 20% v/v DMSO, not more than 25% v/v DMSO, or not more than 30% v/v DMSO.

A buffer for amplification can comprise about 0.5% v/v DMSO, about 1% v/v DMSO, about 2% v/v DMSO, about 3% v/v DMSO, about 4% v/v DMSO, about 5% v/v DMSO, about 6% v/v DMSO, about 7% v/v DMSO, about 8% v/v DMSO, about 9% v/v DMSO, about 10% v/v DMSO, about 15% v/v DMSO, about 20% v/v DMSO, about 25% v/v DMSO, or about 30% v/v DMSO.

A buffer for amplification can comprise between 0.5% v/v DMSO and 30% v/v DMSO, between 0.5% v/v DMSO and 25% v/v DMSO, between 0.5% v/v DMSO and 20% v/v DMSO, between 0.5% v/v DMSO and 15% v/v DMSO, between 0.5% v/v DMSO and 10% v/v DMSO, between 0.5% v/v DMSO and 9% v/v DMSO, between 0.5% v/v DMSO and 8% v/v DMSO, between 0.5% v/v DMSO and 7% v/v DMSO, between 0.5% v/v DMSO and 6% v/v DMSO, between 0.5% v/v DMSO and 5% v/v DMSO, between 0.5% v/v DMSO and 4% v/v DMSO, between 0.5% v/v DMSO and 3% v/v DMSO, between 0.5% v/v DMSO and 2% v/v DMSO, between 0.5% v/v DMSO and 1% v/v DMSO, between 1% v/v DMSO and 30% v/v DMSO, between 1% v/v DMSO and 25% v/v DMSO, between 1% v/v DMSO and 20% v/v DMSO, between 1% v/v DMSO and 15% v/v DMSO, between 1% v/v DMSO and 10% v/v DMSO, between 1% v/v DMSO and 9% v/v DMSO, between 1% v/v DMSO and 8% v/v DMSO, between 1% v/v DMSO and 7% v/v DMSO, between 1% v/v DMSO and 6% v/v DMSO, between 1% v/v DMSO and 5% v/v DMSO, between 1% v/v DMSO and 4% v/v DMSO, between 1% v/v DMSO and 3% v/v DMSO, between 1% v/v DMSO and 2% v/v DMSO, between 2% v/v DMSO and 30% v/v DMSO, between 2% v/v DMSO and 25% v/v DMSO, between 2% v/v DMSO and 20% v/v DMSO, between 2% v/v DMSO and 15% v/v DMSO, between 2% v/v DMSO and 10% v/v DMSO, between 2% v/v DMSO and 9% v/v DMSO, between 2% v/v DMSO and 8% v/v DMSO, between 2% v/v DMSO and 7% v/v DMSO, between 2% v/v DMSO and 6% v/v DMSO, between 2% v/v DMSO and 5% v/v DMSO, between 2% v/v DMSO and 4% v/v DMSO, between 2% v/v DMSO and 3% v/v DMSO, between 3% v/v DMSO and 30% v/v DMSO, between 3% v/v DMSO and 25% v/v DMSO, between 3% v/v DMSO and 20% v/v DMSO, between 3% v/v DMSO and 15% v/v DMSO, between 3% v/v DMSO and 10% v/v DMSO, between 3% v/v DMSO and 9% v/v DMSO, between 3% v/v DMSO and 8% v/v DMSO, between 3% v/v DMSO and 7% v/v DMSO, between 3% v/v DMSO and 6% v/v DMSO, between 3% v/v DMSO and 5% v/v DMSO, between 3% v/v DMSO and 4% v/v DMSO, between 4% v/v DMSO and 30% v/v DMSO, between 4% v/v DMSO and 25% v/v DMSO, between 4% v/v DMSO and 20% v/v DMSO, between 4% v/v DMSO and 15% v/v DMSO, between 4% v/v DMSO and 10% v/v DMSO, between 4% v/v DMSO and 9% v/v DMSO, between 4% v/v DMSO and 8% v/v DMSO, between 4% v/v DMSO and 7% v/v DMSO, between 4% v/v DMSO and 6% v/v DMSO, between 4% v/v DMSO and 5% v/v DMSO, between 5% v/v DMSO and 30% v/v DMSO, between 5% v/v DMSO and 25% v/v DMSO, between 5% v/v DMSO and 20% v/v DMSO, between 5% v/v DMSO and 15% v/v DMSO, between 5% v/v DMSO and 10% v/v DMSO, between 5% v/v DMSO and 9% v/v DMSO, between 5% v/v DMSO and 8% v/v DMSO, between 5% v/v DMSO and 7% v/v DMSO, between 5% v/v DMSO and 6% v/v DMSO, between 6% v/v DMSO and 30% v/v DMSO, between 6% v/v DMSO and 25% v/v DMSO, between 6% v/v DMSO and 20% v/v DMSO, between 6% v/v DMSO and 15% v/v DMSO, between 6% v/v DMSO and 10% v/v DMSO, between 6% v/v DMSO and 9% v/v DMSO, between 6% v/v DMSO and 8% v/v DMSO, between 6% v/v DMSO and 7% v/v DMSO, between 7% v/v DMSO and 30% v/v DMSO, between 7% v/v DMSO and 25% v/v DMSO, between 7% v/v DMSO and 20% v/v DMSO, between 7% v/v DMSO and 15% v/v DMSO, between 7% v/v DMSO and 10% v/v DMSO, between 7% v/v DMSO and 9% v/v DMSO, between 7% v/v DMSO and 8% v/v DMSO, between 8% v/v DMSO and 30% v/v DMSO, between 8% v/v DMSO and 25% v/v DMSO, between 8% v/v DMSO and 20% v/v DMSO, between 8% v/v DMSO and 15% v/v DMSO, between 8% v/v DMSO and 10% v/v DMSO, between 8% v/v DMSO and 9% v/v DMSO, between 9% v/v DMSO and 30% v/v DMSO, between 9% v/v DMSO and 25% v/v DMSO, between 9% v/v DMSO and 20% v/v DMSO, between 9% v/v DMSO and 15% v/v DMSO, between 9% v/v DMSO and 10% v/v DMSO, between 10% v/v DMSO and 30% v/v DMSO, between 10% v/v DMSO and 25% v/v DMSO, between 10% v/v DMSO and 20% v/v DMSO, between 10% v/v DMSO and 15% v/v DMSO, between 15% v/v DMSO and 30% v/v DMSO, between 15% v/v DMSO and 25% v/v DMSO, between 15% v/v DMSO and 20% v/v DMSO, between 20% v/v DMSO and 30% v/v DMSO, between 20% v/v DMSO and 25% v/v DMSO, or between 25% v/v DMSO and 30% v/v DMSO.

In some cases, a buffer for amplification can have a specified pH. A buffer for amplification can have a pH of about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some cases, a buffer can have a pH of between 6.8 and 7.0, between 6.9 and 7.1, between 7.0 and 7.2, between 7.1 and 7.3, between 7.2 and 7.4, between 7.3 and 7.5, between 7.4 and 7.6, between 7.5 and 7.7, between 7.6 and 7.8, between 7.7 and 7.9, between 7.8 and 8.0, between 7.9 and 8.1, between 8.0 and 8.2, between 8.1 and 8.3, between 8.2 and 8.4, between 8.3 and 8.5, between 8.4 and 8.6, between 8.5 and 8.7, between 8.6 and 8.8, between 8.7 and 8.9, between 8.8 and 9.0, or between 8.9 and 9.1.

Components can comprise a circular nucleic acid primer, as described herein.

Components can comprise an oligonucleotide, such as an oligonucleotide attached to the capture molecule as described herein.

Components can comprise one or more nucleotide triphosphates. Nucleoside triphosphates can comprise DNA nucleoside triphosphates (dNTPs) or RNA nucleoside triphosphates. In some cases, NTP components can comprise adenosine triphosphate (ATP), deoxyadenosine triphosphate (dATP), guanosine triphosphate (GTP), deoxyguanosine triphosphate (dGTP), cytidine triphosphate (CTP), deoxycytidine triphosphate (dCTP), thymidine triphosphate (TTP), deoxythymidine triphosphate (dTTP), uridine triphosphate (UTP), and/or deoxyuridine triphosphate (dUTP). In some cases, a mixture of NTP molecules can be provided. In some cases, a mixture of NTP molecules can comprise ATP, GTP, CTP, and UTP. Other NTP molecules can comprise a tautomer of dATP, a tautomer of dGTP, a tautomer of dCTP, a tautomer of dATP, a tautomer of dUTP.

In some cases, a mixture of NTP molecules can comprise ATP, GTP, CTP, and TTP. In some cases, a mixture of NTP molecules can comprise dATP, dGTP, dCTP, and dUTP. In some cases, a mixture of NTP molecules can comprise dATP, dGTP, dCTP, and dTTP. In some cases, other NTP molecules can be included in the components for an RCA reaction.

In some cases, components can comprise at least 100 μM of each of dATP, dGTP, dCTP, and dTTP; at least 200 μM of each of dATP, dGTP, dCTP, and dTTP; at least 300 μM of each of dATP, dGTP, dCTP, and dTTP; at least 400 μM of each of dATP, dGTP, dCTP, and dTTP; at least 600 μM of each of dATP, dGTP, dCTP, and dTTP; at least 600 μM of each of dATP, dGTP, dCTP, and dTTP; or at least 700 μM of each of dATP, dGTP, dCTP, and dTTP.

In some cases, components can comprise not more than 100 μM of each of dATP, dGTP, dCTP, and dTTP; not more than 200 μM of each of dATP, dGTP, dCTP, and dTTP; not more than 300 μM of each of dATP, dGTP, dCTP, and dTTP; not more than 400 μM of each of dATP, dGTP, dCTP, and dTTP; not more than 500 μM of each of dATP, dGTP, dCTP, and dTTP; not more than 600 μM of each of dATP, dGTP, dCTP, and dTTP, or not more than 700 μM of each of dATP, dGTP, dCTP, and dTTP.

In some cases, components can comprise between 100 and 700 μM of each of dATP, dGTP, dCTP, and dTTP; between 100 and 600 μM of each of dATP, dGTP, dCTP, and dTTP; between 100 and 500 μM of each of dATP, dGTP, dCTP, and dTTP; between 100 and 400 μM of each of dATP, dGTP, dCTP, and dTTP; between 100 and 300 μM of each of dATP, dGTP, dCTP, and dTTP; between 100 and 200 μM of each of dATP, dGTP, dCTP, and dTTP; between 200 and 700 μM of each of dATP, dGTP, dCTP, and dTTP; between 200 and 600 μM of each of dATP, dGTP, dCTP, and dTTP; between 200 and 500 μM of each of dATP, dGTP, dCTP, and dTTP; between 200 and 400 μM of each of dATP, dGTP, dCTP, and dTTP; between 200 and 300 μM of each of dATP, dGTP, dCTP, and dTTP; between 300 and 700 μM of each of dATP, dGTP, dCTP, and dTTP; between 300 and 600 μM of each of dATP, dGTP, dCTP, and dTTP; between 300 and 500 μM of each of dATP, dGTP, dCTP, and dTTP; between 300 and 400 μM of each of dATP, dGTP, dCTP, and dTTP; between 400 and 700 μM of each of dATP, dGTP, dCTP, and dTTP; between 400 and 600 μM of each of dATP, dGTP, dCTP, and dTTP; between 400 and 500 μM of each of dATP, dGTP, dCTP, and dTTP; between 500 and 700 μM of each of dATP, dGTP, dCTP, and dTTP; between 500 and 600 μM of each of dATP, dGTP, dCTP, and dTTP; or between 600 and 700 μM of each of dATP, dGTP, dCTP, and dTTP.

An RCA reaction can comprise an elongation step. The elongation can comprise a single strand DNA elongation or a single strand RNA elongation. Elongation can comprise adding nucleotides to a single oligonucleotide strand (i.e., an oligonucleotide linked to a capture agent) according to a template nucleic acid strand (i.e., a circular nucleic acid primer). Addition of nucleic acids to the oligonucleotide strand can be mediated by an enzyme, such as a polymerase.

An elongation step can comprise incubating an oligonucleotide (e.g., an oligonucleotide linked to a capture agent, which is fixed to a sample) with PCR components (including for example a polymerase, buffer, a circular nucleic acid primer, and NTPs) at a temperature at which the polymerase can add nucleotides to the oligonucleotide. Nucleotides can be added to form a long chain of nucleotides appended to an end of the oligonucleotide.

An elongation step can occur at a temperature of at least 20° C., at least 25° C., at least 30° C., at least 35° C., at least 37° C., at least 40° C., at least 45° C., at least 50° C., at least 55° C., at least 60° C., at least 65° C., at least 70° C., or at least 75° C.

An elongation step can occur at a temperature of not more than 20° C., not more than 25° C., not more than 30° C., not more than 35° C., not more than 37° C., not more than 40° C., not more than 45° C., not more than 50° C., not more than 55° C., not more than 60° C., not more than 65° C., not more than 70° C., or not more than 75° C.

An elongation step can occur at a temperature of about 20° C., about 25° C., about 30° C., about 35° C., about 37° C., about 40° C., about 45° C., about 50° C., about 55° C., about 60° C., about 65° C., about 70° C., or about 75° C.

An elongation step can occur at a temperature between 20° C. and 75° C., between 20° C. and 70° C., between 20° C. and 65° C., between 20° C. and 60° C., between 20° C. and 55° C., between 20° C. and 50° C., between 20° C. and 45° C., between 20° C. and 40° C., between 20° C. and 35° C., between 20° C. and 30° C., between 20° C. and 25° C., between 25° C. and 75° C., between 25° C. and 70° C., between 25° C. and 65° C., between 25° C. and 60° C., between 25° C. and 55° C., between 25° C. and 50° C., between 25° C. and 45° C., between 25° C. and 40° C., between 25° C. and 35° C., between 25° C. and 30° C., between 30° C. and 75° C., between 30° C. and 70° C., between 30° C. and 65° C., between 30° C. and 60° C., between 30° C. and 55° C., between 30° C. and 50° C., between 30° C. and 45° C., between 30° C. and 40° C., between 30° C. and 35° C., between 35° C. and 75° C., between 35° C. and 70° C., between 35° C. and 65° C., between 35° C. and 60° C., between 35° C. and 55° C., between 35° C. and 50° C., between 35° C. and 45° C., between 35° C. and 40° C., between 40° C. and 75° C., between 40° C. and 70° C., between 40° C. and 65° C., between 40° C. and 60° C., between 40° C. and 55° C., between 40° C. and 50° C., between 40° C. and 45° C., between 45° C. and 75° C., between 45° C. and 70° C., between 45° C. and 65° C., between 45° C. and 60° C., between 45° C. and 55° C., between 45° C. and 50° C., between 50° C. and 75° C., between 50° C. and 70° C., between 50° C. and 65° C., between 50° C. and 60° C., between 50° C. and 55° C., between 55° C. and 75° C., between 55° C. and 70° C., between 55° C. and 65° C., between 55° C. and 60° C., between 60° C. and 75° C., between 60° C. and 70° C., between 60° C. and 65° C., between 65° C. and 75° C., between 65° C. and 70° C., or between 70° C. and 75° C.

An elongation step can last for at least 1 minute, at least 5 minutes, at least 15 minutes, at least 30 minutes, at least 45 minutes, at least 1 hour, at least 1.5 hours, at least 2 hours, at least 2.5 hours, at least 3 hours, at least 3.5 hours, at least 4 hours, at least 4.5 hours, or at least 5 hours.

An elongation step can last for not more than 1 minute, not more than 5 minutes, not more than 15 minutes, not more than 30 minutes, not more than 45 minutes, not more than 1 hour, not more than 1.5 hours, not more than 2 hours, not more than 2.5 hours, not more than 4 hours, not more than 3.5 hours, not more than 4 hours, not more than 4.5 hours, or not more than 5 hours.

An elongation step can last for about 1 minute, about 5 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, or about 5 hours.

An elongation step can last for between 1 minute and 5 hours, between 1 minute and 4.5 hours, between 1 minute and 4 hours, between 1 minute and 3.5 hours, between 1 minute and 3 hours, between 1 minute and 2.5 hours, between 1 minute and 2 hours, between 1 minute and 1.5 hours, between 1 minute and 1 hour, between 1 minute and 45 minutes, between 1 minute and 30 minutes, between 1 minute and 15 minutes, between 1 minute and 5 minutes, between 5 minutes and 5 hours, between 5 minutes and 4.5 hours, between 5 minutes and 4 hours, between 5 minutes and 3.5 hours, between 5 minutes and 3 hours, between 5 minutes and 2.5 hours, between 5 minutes and 2 hours, between 5 minutes and 1.5 hours, between 5 minutes and 1 hour, between 5 minutes and 45 minutes, between 5 minutes and 30 minutes, between 5 minutes and 15 minutes, between 15 minutes and 5 hours, between 15 minutes and 4.5 hours, between 15 minutes and 4 hours, between 15 minutes and 3.5 hours, between 15 minutes and 3 hours, between 15 minutes and 2.5 hours, between 15 minutes and 2 hours, between 15 minutes and 1.5 hours, between 15 minutes and 1 hour, between 15 minutes and 45 minutes, between 15 minutes and 30 minutes, between 30 minutes and 5 hours, between 30 minutes and 4.5 hours, between 30 minutes and 4 hours, between 30 minutes and 3.5 hours, between 30 minutes and 3 hours, between 30 minutes and 2.5 hours, between 30 minutes and 2 hours, between 30 minutes and 1.5 hours, between 30 minutes and 1 hour, between 30 minutes and 45 minutes, between 45 minutes and 5 hours, between 45 minutes and 4.5 hours, between 45 minutes and 4 hours, between 45 minutes and 3.5 hours, between 45 minutes and 3 hours, between 45 minutes and 2.5 hours, between 45 minutes and 2 hours, between 45 minutes and 1.5 hours, between 45 minutes and 1 hour, between 1 hour and 5 hours, between 1 hour and 4.5 hours, between 1 hour and 4 hours, between 1 hour and 3.5 hours, between 1 hour and 3 hours, between 1 hour and 2.5 hours, between 1 hour and 2 hours, between 1 hour and 1.5 hours, between 1.5 hours and 5 hours, between 1.5 hours and 4.5 hours, between 1.5 hours and 4 hours, between 1.5 hours and 3.5 hours, between 1.5 hours and 3 hours, between 1.5 hours and 2.5 hours, between 1.5 hours and 2 hours, between 2 hours and 5 hours, between 2 hours and 4.5 hours, between 2 hours and 4 hours, between 2 hours and 3.5 hours, between 2 hours and 3 hours, between 2 hours and 2.5 hours, between 2.5 hours and 5 hours, between 2.5 hours and 4.5 hours, between 2.5 hours and 4 hours, between 2.5 hours and 3.5 hours, between 2.5 hours and 3 hours, between 3 hours and 5 hours, between 3 hours and 4.5 hours, between 3 hours and 4 hours, between 3 hours and 3.5 hours, between 3.5 hours and 5 hours, between 3.5 hours and 4.5 hours, between 3.5 hours and 4 hours, between 4 hours and 5 hours, between 4 hours and 4.5 hours, or between 4.5 hours and 5 hours.

In some cases, RCA can produce an amplification. In some cases, a higher amplification can indicate a higher number of times a template (i.e., circular nucleic acid primer) has been copied.

Amplification can be quantified as the number of labeled probes which can bind to the amplified oligonucleotide for detection. For example, 10× amplification can comprise amplification that can result in 10 labeled probes binding to one oligonucleotide for detection. In some cases, a higher amplification can indicate a higher number of labeled probes which can bind to an amplified oligonucleotide. A higher number of labeled probes can produce a higher signal which can be detected.

In some cases, RCA can result in at least 2× amplification, at least 5× amplification, at least 10× amplification, at least 20× amplification, at least 30× amplification, at least 40× amplification, at least 50× amplification, at least 60× amplification, at least 70× amplification, at least 80× amplification, at least 90× amplification, at least 100× amplification, at least 200× amplification, at least 300× amplification, at least 400× amplification, at least 500× amplification, at least 600× amplification, at least 700× amplification, at least 800× amplification, at least 900× amplification, at least 1000× amplification, at least 5000× amplification, or at least 10000× amplification.

In some cases, RCA can result in no more than 2× amplification, no more than 5× amplification, no more than 10× amplification, no more than 20× amplification, no more than 30× amplification, no more than 40× amplification, no more than 50× amplification, no more than 60× amplification, no more than 70× amplification, no more than 80× amplification, no more than 90× amplification, no more than 100× amplification, no more than 200× amplification, no more than 300× amplification, no more than 400× amplification, no more than 500× amplification, no more than 600× amplification, no more than 700× amplification, no more than 800× amplification, no more than 900× amplification, no more than 1000× amplification, no more than 5000× amplification, or no more than 10000× amplification.

In some cases, RCA can result in between 2× amplification and 10000× amplification, between 2× and 5000× amplification, between 2× amplification and 1000× amplification, between 2× amplification and 500× amplification, between 2× amplification and 100× amplification, between 2× amplification and 50× amplification, between 2× amplification and 10× amplification, between 10× and 10000× amplification, between 10× and 5000× amplification, between 10× amplification and 1000× amplification, between 10× and 500× amplification, between 10× and 100× amplification, between 10× amplification and 50× amplification, between 50× amplification and 10000× amplification, between 50× amplification and 5000× amplification, between 50× amplification and 1000× amplification, between 50× amplification and 500× amplification, between 50× amplification and 100× amplification, between 100× amplification and 10000× amplification, between 100× amplification and 5000× amplification, between 100× amplification and 1000× amplification, between 100× amplification and 500× amplification, between 500× amplification and 10000× amplification, between 500× amplification and 5000× amplification, between 500× amplification and 1000× amplification, between 1000× amplification and 10000× amplification, between 1000× amplification and 5000× amplification, or between 5000× amplification and 10000× amplification.

### Probes

A probe can be a molecule used for the detection of a biological feature of interest.

After the sample has been bound to the capture agents and an RCA reaction implemented, a method can involve contacting a set of labeled probes with the sample. In some cases, such as where the labeled probes are labeled nucleic acid probes, the contacting can comprise specifically hybridizing the probes to the sample. Herein, the probes can be distinguishably labeled, to produce labeled probe/oligonucleotide duplexes.

In some cases, at least 1, 2, 3, 4, 5, 6, 7, or 8 probes can be applied. In some cases, no more than 1, 2, 3, 4, 5, 6, 7, or 8 probes can be applied. In some cases, between 1 and 8 probes, between 1 and 7 probes, between 1 and 6 probes, between 1 and 5 probes, between 1 and 4 probes, between 1 and 3 probes, between 1 and 2 probes, between 2 and 8 probes, between 2 and 7 probes, between 2 and 6 probes, between 2 and 5 probes, between 2 and 4 probes, between 2 and 3 probes, between 3 and 8 probes, between 3 and 7 probes, between 3 and 6 probes, between 3 and 5 probes, between 3 and 4 probes, between 4 and 8 probes, between 4 and 7 probes, between 4 and 6 probes, between 4 and 5 probes, between 5 and 8 probes, between 5 and 7 probes, between 5 and 6 probes, between 6 and 8 probes, between 6 and 7 probes, or between 7 and 8 probes can be applied.

In some cases, a secondary nucleic acid amplification step, including, but not limited, to hybridization chain reaction, branched DNA (bDNA) amplification, etc., can be performed prior to applying the labeled probes.

In some embodiments, a probe may have a calculated melting temperature (Tₘ) in the range of 15° C. to 70° C. (e.g., 20° C.-60° C. or 35° C.-50° C.) such that the duplexes of the hybridization step have a Tm in the same range. In these embodiments, the Tₘ may be calculated using the IDT oligoanalyzer program (available at IDT's website and described in Owczarzy et al., Nucleic Acids Res. 2008 36: W163-9), for example by using the default settings of 50 mM Na+ and 250 nM oligonucleotide.

A probe can be Tₘ-matched, where the term “Tₘ-matched” can refer to a sequence that has a melting temperature within a defined range, e.g., less than 15° C., less than 10° C. or less than 5° C. of a defined temperature. As would be apparent, the probes may be labeled at the 5′ end, the 3′ end or anywhere in between. In some embodiments, a probe can be specifically cleavable. For example, a probe can contain a cleavable linker (e.g., a photo- or chemically-cleavable linker).

A Tₘ of a probe-amplified oligonucleotide duplex can be about 10° C., about 15° C., about 20° C., about 25° C., or about 30° C. A Tₘ of a probe-amplified oligonucleotide duplex can be at least 10° C., at least 15° C., at least 20° C., at least 25° C., or at least 30° C. A Tₘ of a probe-amplified oligonucleotide duplex can be no more than 10° C., no more than 15° C., no more than 20° C., no more than 25° C., or no more than 30° C. A Tₘ of a probe-amplified oligonucleotide duplex can be between 10° C. and 30° C., between 10° C. and 25° C., between 10° C. and 20° C., between 10° C. and 15° C., between 15° C. and 30° C., between 15° C. and 25° C., between 15° C. and 20° C., between 20° C. and 30° C., between 20° C. and 25° C., or between 25° C. and 30° C.

In some cases, a probe can be incubated on the sample to allow hybridization to the sample. A probe can be incubated at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., or about 55° C.

A probe can be incubated on a sample for at least 10 seconds, at least 15 seconds, at least 30 seconds, at least 1 minute, at least 2 minutes, at least 3 minutes, at least 4 minutes, at least 5 minutes, at least 10 minutes, or at least 15 minutes. In some cases, a wash can last for less than 10 seconds.

A probe can be incubated on a sample for up to 10 seconds, up to 15 seconds, up to 30 seconds, up to 1 minute, up to 2 minutes, up to 3 minutes, up to 4 minutes, up to 5 minutes, up to 10 minutes, or up to 15 minutes. In some cases, a wash can last for more than 15 minutes.

A probe can be incubated on a sample for between 10 seconds and 15 minutes, between 10 seconds and 10 minutes, between 10 seconds and 5 minutes, between 10 seconds and 1 minute, between 10 seconds and 30 seconds, between 30 seconds and 15 minutes, between 30 seconds and 10 minutes, between 30 seconds and 5 minutes, between 30 seconds and 1 minute, between 1 minute and 15 minutes, between 1 minute and 10 minutes, between 1 minute and 5 minutes, between 5 minutes and 15 minutes, between 5 minutes and 10 minutes, or between 10 minutes and 15 minutes.

### Labels

A label can be a detectable molecule conjugated or linked to a probe. Labels linked to probes applied during a single iteration can be distinguishable from each other, such that those probes are distinguishably labeled, as described above.

Distinguishably labeled probes can comprise distinguishable labels. Such labels can be distinguished on the basis of excitation wavelength, emission wavelength, intensity, or some other property. In some embodiments, a set of labeled probes which are fluorescently labeled can comprise probes labeled with one or more distinguishable fluorescent labeled pairs.

Suitable distinguishable fluorescent label pairs useful in the subject methods include Cy3 and Cy5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), Alexafluor 750, POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002), Ried et al. (Proc. Natl. Acad. Sci. 1992: 89: 1388-1392) and Tanke et al. (Eur. J. Hum. Genet. 1999 7:2-11) and others. In some embodiments three or four distinguishable dyes may be used. Specific fluorescent dyes of interest include: xanthene dyes, e.g., fluorescein and rhodamine dyes, such as fluorescein isothiocyanate (FITC), 6 carboxyfluorescein (commonly known by the abbreviations FAM and F), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g., Cy3, Cy5 and Cy7 dyes; coumarins, e.g., umbelliferone; benzamide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, Napthofluorescein, Texas Red, Cy3, and Cy5, etc. As noted above, within each sub-set of probes, the fluorophores may be chosen so that they are distinguishable, i.e., independently detectable, from one another, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same area of the section).

The label may be a pro-fluorophore, a secondary activatable fluorophore, a fluorescent protein, a visible stain, a polychromatic barcode, a mass tag (e.g., an isotope or a polymer of a defined size), a structural tags for label-free detection, a radio sensitive tag (activated by THz camera) a radioactive tag or an absorbance tag that only absorbs light at a specific frequency for example. In some embodiments, an oligonucleotide may deliver an enzyme that delivers a fluorophore or there may be an enzymatic amplification of signal. In some cases, detectable signal of a label can be generated in some cases by fluorescence resonance energy transfer (FRET), Raman spectroscopy, infrared detection, or magnetic/electrical detection.

In some cases, the label can comprise an enzyme. In such cases, the enzyme can mediate the deposition of a detectable of substance on a biological sample. In some cases, this deposition of a detectable substance can constitute signal amplification. For example, a label can comprise horseradish peroxidase or a synthetic enzyme engineered to have properties similar to those of horseradish peroxidase, which can mediate tyramide-signal amplification. For example, an enzyme can mediate an oxidation-reduction reaction using a substrate such as hydrogen peroxide to mediate the deposition of a dye or label, such as a dye or label conjugated tyramide, to the surface for detection.

### Linkers

In compositions herein, different molecules can be connected via one or more linkers. For example, the capture agent can be attached to the oligonucleotide via a linker. As another example, the probe can be attached to the probe via a linker.

Linkers can comprise a direct bond or an atom such as oxygen or sulfur, a unit such as NR1, C(O), C(O)NH, SO, SO2, SO2NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO2, N(R1)2, C(O), cleavable linking group, substituted or unsubstituted aryl, substituted or unsubstituted heteroaryl, substituted or unsubstituted heterocyclic; where R1 is hydrogen, acyl, aliphatic or substituted aliphatic.

In some cases, the linker can be a nucleic acid linker. A “nucleic acid linker” can be a nucleic acid that connects two parts of a compound, e.g., an affinity molecule to a label moiety. A nucleic acid linker can be single-stranded, fully double-stranded, or partially double-stranded. A nucleic acid linker can be any length. For example, a nucleic acid linker can be from 1 nucleotide to about 100 nucleotides in length. When the nucleic acid linker is double-stranded, the linker can comprise a double stranded region of about 6 to about 100 consecutive base pairs. However, the duplex region can be interrupted by one or more single-stranded regions in one or both of the strands of the duplex. Further, a double-stranded nucleic acid linker can comprise a single-stranded overhang on one or both ends of the double-stranded region. Moreover, a nucleic acid linker can comprise one or more nucleic acid modifications described herein. A nucleic acid linker can be attached to a compound by a non-nucleic acid linker.

In some cases, a linker can be a “non-nucleic acid linker” which can be any linker that is not a nucleic acid linker.

A linker can link molecules covalently or non-covalently. Accordingly, in some embodiments, the capture agent and the oligonucleotide can be covalently linked together using a non-nucleic acid linker. For example, the capture agent and the oligonucleotide can be covalently linked together via a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between COOH and —NH₂ directly on affinity molecule and nucleic acid), two peptide bonds on a spacer (from cross-linking of two —NH₂ groups), triazole bond (from “click” reaction), a phosphodiester linkage, a phosphothioate linkage, and any combination thereof. In another example, the probe and the label can be covalently linked together via a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between —COOH and —NH₂ directly on affinity molecule and nucleic acid), two peptide bonds on a spacer (from cross-linking of two —NH₂ groups), triazole bond (from “click” reaction), a phosphodiester linkage, a phosphothioate linkage, and any combination thereof.

### Methods

A biological sample can be procured or prepared prior to or as part of methods described herein. Non-limiting examples of biological samples can include tissue, cells, or organs

In some cases, a protein blocking agent can be applied to the sample prior to the application of the capture agent.

A capture agent (or a plurality of capture agents) can be incubated on the sample. The capture agents can be linked to oligonucleotides, such that each capture agent is linked to a different oligonucleotide, as described herein. In some cases, one capture agent at a time can be incubated with the sample at the same time. In some cases, 2, 3, 4, 5, 6, 7, 8, or more capture agents can be incubated with the sample at the same time. In some cases, all capture agents can be incubated with the sample at the same time.

In some cases, a capture agent can be incubated at about 4° C., about 10° C., about 15° C., about 20° C., about 25° C., about 30° C., about 35° C., about 40° C., or about 45° C. In some cases, a capture agent can be incubated at at least 4° C., at least 10° C., at least 15° C., at least 20° C., at least 25° C., at least 30° C., at least 35° C., at least 40° C., or at least 45° C. In some cases, a capture agent can be incubated at not more than 4° C., not more than 10° C., not more than 15° C., not more than 20° C., not more than 25° C., not more than 30° C., not more than 35° C., not more than 40° C., or not more than 45° C. In some cases, a capture agent can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

A capture agent can be incubated for about 30 minutes, about 1 hour, about 2 hours, about 3 hours, about 4 hours, about 5 hours, or about 6 hours. In some cases, a capture agent can be incubated for at least 30 minutes, at least 1 hour, at least 2 hours, at least 3 hours, at least 4 hours, at least 5 hours, or at least 6 hours. In some cases, a capture agent can be incubated for not more than 30 minutes, not more than 1 hour, not more than 2 hours, not more than 3 hours, not more than 4 hours, not more than 5 hours, or not more than 6 hours. In some cases, a capture agent can be incubated for between 30 minutes and 6 hours, between 30 minutes and 5 hours, between 30 minutes and 4 hours, between 30 minutes and 3 hours, between 30 minutes and 2 hours, between 30 minutes and 1 hour, between 1 hour and 6 hours, between 1 hour and 5 hours, between 1 hour and 4 hours, between 1 hour and 3 hours, between 1 hour and 2 hours, between 2 hours and 6 hours, between 2 hours and 5 hours, between 2 hours and 4 hours, between 2 hours and 3 hours, between 3 hours and 6 hours, between 3 hours and 5 hours, between 3 hours and 4 hours, between 4 hours and 6 hours, between 4 hours and 5 hours, or between 5 hours and 6 hours.

Following incubation with the capture agent, the sample can be washed to remove excess capture agent. Washing can comprise applying a buffer to the sample for an amount of time followed by removal of the buffer. In some cases, washing can comprise gentle agitation, such as by swirling, shaking, swinging, or rocking the sample. Washing can comprise applying at least at least 100 at least 500 at least 1 mL, at least 5 mL, at least 10 mL, at least 20 mL, at least 30 mL, at least 40 mL, or at least 50 mL buffer to the sample. Washing can comprise applying no more than 50 no more than 100 no more than 500 no more than 1 mL, no more than 5 mL, no more than 10 mL, no more than 20 mL, no more than 30 mL, no more than 40 mL, or no more than 50 mL buffer to the sample. In some cases, washing can comprise applying between 50 and 50 mL, between 50 and 40 mL, between 50 and 30 mL, between 50 and 20 mL, between 50 and 10 mL, between 50 and 5 mL, between 50 μL, and 1 mL, between 50 μL, and 500 μL, between 50 μL, and 100 μL, between 100 μL, and 50 mL, between 100 μL, and 40 mL, between 100 μL, and 30 mL, between 100 μL and 20 mL, between 100 μL and 10 mL, between 100 μL and 5 mL, between 100 μL and 1 mL, between 100 μL and 500 μL, between 500 μL and 50 mL, between 500 μL and 40 mL, between 500 μL and 30 mL, between 500 μL and 20 mL, between 500 μL and 10 mL, between 500 μL and 5 mL, between 500 μL and 1 mL, between 1 mL and 50 mL, between 1 mL and 40 mL, between 1 mL and 30 mL, between 1 mL and 20 mL, between 1 mL and 10 mL, between 1 mL and 5 mL, between 5 mL and 50 mL, between 5 mL and 40 mL, between 5 mL and 30 mL, between 5 mL and 20 mL, between 5 mL and 10 mL, between 10 mL and 50 mL, between 10 mL and 40 mL, between 10 mL and 30 mL, between 10 mL and 20 mL, between 20 mL and 50 mL, between 20 mL and 40 mL, between 20 mL and 30 mL, between 30 mL and 50 mL, between 30 mL and 40 mL, or between 40 mL and 50 mL buffer to the sample. Wash buffer can be any acceptable buffer. In some cases, wash buffer can be for example a same buffer that the capture agent is in, or another buffer, such as PBS, PBS-T, TBS, or TBS-T. The washing step can last for at least 10 seconds, at least 30 seconds, at least 1 minute, at least 2 minutes, at least 3 minutes, at least 4 minutes, at least 5 minutes, at least 10 minutes, or at least 15 minutes. The washing step can last for up to 10 seconds, up to 30 seconds, up to 1 minute, up to 2 minutes, up to 3 minutes, up to 4 minutes, up to 5 minutes, up to 10 minutes, or up to 15 minutes. The washing step can last between 10 seconds and 15 minutes, between 10 seconds and 10 minutes, between 10 seconds and 5 minutes, between 10 seconds and 30 seconds, between 30 seconds and 15 minutes, between 30 seconds and 10 minutes, between 30 seconds and 5 minutes, between 30 seconds and 1 minute, between 1 minute and 15 minutes, between 1 minute and 10 minutes, between 1 minute and 5 minutes, between 5 minutes and 15 minutes, between 5 minutes and 10 minutes, or between 1 minutes and 15 minutes. The washing step can be performed 1, 2, 3, 4, 5, or more times.

The capture agent can be cross-linked to the sample. Such cross-linking can prevent the capture agent from disassociating during subsequent steps. This crosslinking step may be done using any amine-to-amine crosslinker (e.g. formaldehyde, paraformaldehyde, disuccinimiyllutarate, N-hydroxysuccinimide (NETS), or another reagent of similar action) although a variety of other chemistries can be used to cross-link the capture agent to the sample if desired.

In some cases, a nucleic acid blocking agent can be applied to the sample prior to the application of the circular nucleic acid primer. Any acceptable nucleic acid blocking agent can be used in this step, such as salmon sperm DNA or another commercially available product.

In some cases, a nucleic acid blocking agent can be incubated at about 4° C., about 10° C., about 15° C., about 20° C., about 25° C., about 30° C., about 35° C., about 40° C., or about 45° C. In some cases, a nucleic acid blocking agent can be incubated at at least 4° C., at least 10° C., at least 15° C., at least 20° C., at least 25° C., at least 30° C., at least 35° C., at least 40° C., or at least 45° C. In some cases, a nucleic acid blocking agent can be incubated at not more than 4° C., not more than 10° C., not more than 15° C., not more than 20° C., not more than 25° C., not more than 30° C., not more than 35° C., not more than 40° C., or not more than 45° C. In some cases, a nucleic acid blocking agent can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

In some cases, the blocking step can last for about 10 minutes, about 20 minutes, about 30 minutes, about 40 minutes, about 50 minutes, or about 60 minutes. In some cases, the blocking step can last for at least 10 minutes, at least 20 minutes, at least 30 minutes, at least 40 minutes, at least 50 minutes, or at least 60 minutes. In some cases, the blocking step can last for not more than 10 minutes, not more than 20 minutes, not more than 30 minutes, not more than 40 minutes, not more than 50 minutes, or not more than 60 minutes. In some cases, the blocking step can last for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

The circular nucleic acid primer (e.g., a padlock probe) can be applied to the sample. The circular nucleic acid primer can be incubated on the sample such that the circular nucleic acid primer can hybridize to the oligonucleotide.

In some cases, a circular nucleic acid primer can be incubated at about 4° C., about 10° C., about 15° C., about 20° C., about 25° C., about 30° C., about 35° C., about 40° C., or about 45° C. In some cases, a circular nucleic acid primer can be incubated at at least 4° C., at least 10° C., at least 15° C., at least 20° C., at least 25° C., at least 30° C., at least 35° C., at least 40° C., or at least 45° C. In some cases, a circular nucleic acid primer can be incubated at not more than 4° C., not more than 10° C., not more than 15° C., not more than 20° C., not more than 25° C., not more than 30° C., not more than 35° C., not more than 40° C., or not more than 45° C. In some cases, a circular nucleic acid primer can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

In some cases, a circular nucleic acid primer can be incubated on the sample for about 10 minutes, about 20 minutes, about 30 minutes, about 40 minutes, about 50 minutes, or about 60 minutes. In some cases, a circular nucleic acid primer can be incubated on the sample for at least 10 minutes, at least 20 minutes, at least 30 minutes, at least 40 minutes, at least 50 minutes, or at least 60 minutes. In some cases, a circular nucleic acid primer can be incubated on the sample for not more than 10 minutes, not more than 20 minutes, not more than 30 minutes, not more than 40 minutes, not more than 50 minutes, or not more than 60 minutes. In some cases, a circular nucleic acid primer can be incubated on the sample for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

In some cases, after the circular nucleic acid primer is applied to the sample and incubated, the sample can be washed, e.g., to remove excess primer. Washing can be for example as described above.

In some cases, the circular nucleic acid probe can be ligated. For example, in the case, where the circular nucleic acid probe is linear prior to hybridizing with the oligonucleotide, it can be ligated prior to the RCA reaction. Ligation can be performed in a ligation buffer. Ligase (e.g., T4 DNA ligase or other DNA or nucleotide ligase) can be applied to facilitate the ligation. Ligation can be performed in some cases at the same time as the blocking step. In some cases, ligation can be performed at the same time as the circular nucleic acid primer is incubated on the sample for hybridization. In some cases, ligation, hybridization, and blocking can be performed at the same time.

In some cases, a ligase can be incubated at about 4° C., about 10° C., about 15° C., about 20° C., about 25° C., about 30° C., about 35° C., about 40° C., or about 45° C. In some cases, a ligase can be incubated at at least 4° C., at least 10° C., at least 15° C., at least 20° C., at least 25° C., at least 30° C., at least 35° C., at least 40° C., or at least 45° C. In some cases, a ligase can be incubated at not more than 4° C., not more than 10° C., not more than 15° C., not more than 20° C., not more than 25° C., not more than 30° C., not more than 35° C., not more than 40° C., or not more than 45° C. In some cases, a ligase can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

In some cases, ligase can be incubated on the sample for about 10 minutes, about 20 minutes, about 30 minutes, about 40 minutes, about 50 minutes, or about 60 minutes. In some cases, ligase can be incubated on the sample for at least 10 minutes, at least 20 minutes, at least 30 minutes, at least 40 minutes, at least 50 minutes, or at least 60 minutes. In some cases, a ligase can be incubated on the sample for not more than 10 minutes, not more than 20 minutes, not more than 30 minutes, not more than 40 minutes, not more than 50 minutes, or not more than 60 minutes. In some cases, ligase can be incubated on the sample for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

The circular nucleic acid primer can be washed (e.g., to remove excess primer). Washing can be performed for example as described above.

An RCA reaction can be performed to elongate the oligonucleotide using the circular nucleic acid primer as a template. RCA reaction can comprise incubating the sample with reagents for RCA. For example, a sample can be incubated with BSA, a polymerase (e.g., Phi29 polymerase or another polymerase), dNTPs, and a buffer appropriate for the chosen polymerase (e.g., Phi29 polymerase buffer if Phi29 is the selected polymerase).

The sample can be washed after the RCA reaction (e.g., to remove excess reagent, such as excess polymerase or excess dNTPs). Washing can be performed for example as described above.

An RCA reaction can be incubated at about 37° C. In some cases, an RCA reaction can be incubated at about 20° C., about 25° C., about 30° C., about 35° C., about 40° C., or about 45° C. In some cases, an RCA reaction can be incubated at at least 20° C., at least 25° C., at least 30° C., at least 35° C., at least 40° C., or at least 45° C. In some cases, an RCA reaction can be incubated at not more than 20° C., not more than 25° C., not more than 30° C., not more than 35° C., not more than 40° C., or not more than 45° C. In some cases, an RCA reaction can be incubated between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

Labeled probes can be incubated with the elongated oligonucleotide in the presence of DMSO. DMSO can regulate hybridization of the probes to the amplified oligonucleotide, and in some cases, DMSO can have the effect of uncompacting or uncoiling the elongated oligonucleotide to help to facilitate binding of the probes.

Probes can be applied to the sample. Application of probes can be performed for example by pipetting, wiping, pouring, dropping, or otherwise introducing a solution containing the probes to the sample, such that the probes have the opportunity to contact the elongated oligonucleotide.

Probes can be applied to a sample in solution, for example in a buffer described herein. Probes can be applied in a volume sufficient to cover the area of the sample in contact with a capture agent having an elongated oligonucleotide. In some cases, probes can be applied in a volume sufficient to cover the entire sample. In some cases, at least 5 μL, at least 10 μL, at least 15 μL, at least 20 μL, at least 30 μL, at least 40 μL, at least 50 μL, at least 60 μL, at least 70 μL, at least 80 μL, at least 90 μL, at least 100 μL, at least 200 μL, at least 300 μL, at least 400 μL, or at least 500 μL, of probes can be applied to a sample. In some cases, no more than 5 μL, no more than 10 μL, no more than 15 μL, no more than 20 μL, more than 30 μL, no more than 40 μL, no more than 50 μL, no more than 60 μL, no more than 70 μL, no more than 80 μL, no more than 90 μL, no more than 100 μL, no more than 200 μL, no more than 300 μL, no more than 400 μL, no more than 500 μL of probes can be applied to a sample. In some cases, between 5 μL and 500 μL, between 50 μL and 500 μL, between 100 μL and 500 μL, between 200 μL and 500 μL, between 300 μL and 500 μL, between 400 μL and 500 μL, between 5 μL and 400 μL, between 50 μL and 400 μL, between 100 μL and 400 μL, between 200 μL and 400 μL, between 300 μL and 400 μL, between 5 μL and 300 μL, between 50 μL and 300 μL, between 100 μL and 300 μL, between 200 μL and 300 μL, between 5 μL and 200 μL, between 50 μL and 200 μL, between 100 μL and 200 μL, between 5 μL and 100 μL, between 50 μL and 100 μL, or between 5 μL and 50 μL of probes can be applied to a sample.

Probes applied to a sample can be in solution, for example in a buffer. Probes can be present in the solution at a concentration of at least

After the probes are applied to the sample such that they are associated with a biological feature of interest of the sample via the capture agent and elongated oligonucleotide, the probes can be read, or detected, in order to identify and/or quantify the biological feature of interest. A plurality of probes can be associated with each biological feature of interest, thereby amplifying the signal compared with other methods. Reading can be performed as described below.

### Reading

A sample can be read to determine the binding pattern for one or more of the probes. In some cases, a sample can be read to determine the binding pattern for each of the probes. The binding pattern of the probes can indicate spatial information of an oligonucleotide and conjugated capture agent, which can in turn indicate spatial information of a biological feature of interest.

After the sample has been washed to remove any labeled probes that have not hybridized, the method can comprise reading the sample to obtain an image from which the binding pattern for each of the sub-set of probes hybridized in the prior step can be determined. This step may be done using any convenient reading method and, in some embodiments, e.g., hybridization of the different probes can be separately read using a fluorescence microscope equipped with an appropriate filter for each fluorophore, or by using dual or triple band-pass filter sets to observe multiple fluorophores (see, e.g., U.S. Pat. No. 5,776,688).

In some cases, each biological feature of interest associated with a label at the same time another biological feature of interest is associated with a label can be read during the same iteration. Labels read during the same iteration can be different. Two labels can be considered different if they are distinguishable from each other when detected using the reading medium. For example, two fluorescent molecules can be considered different if when imaged using a microscope, their signals are differentiable from each other, e.g. by excitation wavelength, emission wavelength, intensity, or some other property.

Each reading can produce an image of the sample showing the pattern of binding of a sub-set of probes. In some embodiments, the method may further comprise analyzing, comparing or overlaying, at least two of the images. In some embodiments, the method may further comprise overlaying all of the images to produce an image showing the pattern of binding of all of the capture agents to the sample. The image analysis module used may transform the signals from each fluorophore to produce a plurality of false color images. The image analysis module may overlay the plurality of false color images (e.g., superimpose the false colors at each pixel) to obtain a multiplexed false color image. Multiple images (e.g., unweighted or weighted) may be transformed into a single false color, e.g., to represent a biological feature of interest characterized by the binding of a specific capture agent. False colors may be assigned to specific capture agents or combinations of capture agents, based on manual input from the user. In certain aspects, the image may comprise false colors relating only to the intensities of labels associated with a feature of interest, such as in the nuclear compartment. The image analysis module may further be configured to adjust (e.g., normalize) the intensity and/or contrast of signal intensities or false colors, to perform a convolution operation (such as blurring or sharpening of the intensities or false colors), or perform any other suitable operations to enhance the image. The image analysis module may perform any of the above operations to align pixels obtained from successive images and/or to blur or smooth intensities or false colors across pixels obtained from successive images.

In some embodiments, images of the sample may be taken at different focal planes, in the z direction. These optical sections can be used to reconstruct a three-dimensional image of the sample. Optical sections may be taken using confocal microscopy, although other methods are known. The image analysis method may be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

### Inactivation and Removal

Labels can be inactivated or removed. Inactivation or removal can allow for multiplexing of the method, such that a greater plurality of biological features of interest can be detected than without an inactivation or removal step.

After reading the sample, the method may comprise inactivating or removing the labels that are associated with (i.e., hybridized to) the amplified oligonucleotide, leaving the plurality of capture agents and their associated amplified oligonucleotides still bound to the sample. The labels that are associated the sample may be removed or inactivated by a variety of methods including, but not limited to, denaturation (in which case the label and the probe in its entirety can be released and can be washed away), by cleaving a linkage in the probe (in which case the label and part of the probe can be released and can be washed away), by cleaving both the probe and the amplified oligonucleotide to which the probe is hybridized (to release a fragment that can be washed away), by cleaving the linkage between the probe and the label (in which case the label can be released and can be washed away and can be washed away), by cleaving the amplified oligonucleotide such as by using a restriction enzyme (in which case the amplified oligonucleotide and the labeled probe can be washed away), or by inactivating the label itself (e.g., by breaking a bond in the label, thereby preventing the label from producing a signal, or by introducing a quencher to the label to prevent detection of a signal). In acceptable removal methods such as the ones provided, the unhybridized amplified oligonucleotides that are attached to the other antibodies (e.g., antibodies bound to biological features of interest not yet detected) are intact and free to hybridize to the set of labeled probes used in the next cycle. In some embodiments, fluorescence may be inactivated by light-based bleaching, peroxide-based bleaching, or cleavage of a fluorophore linked to a nucleotide through a cleavable linker (e.g., using TCEP as a cleaving reagent).

In some embodiments, the removing step is done by removing the hybridized probes from the sample by denaturation, leaving the other capture agents (i.e., the capture agents that are not hybridized to a probe) and their associated oligonucleotides still bound to the sample. In other embodiments, the removing step is not done by removing the hybridized probes from the sample by denaturation, leaving the other capture agents (i.e., the capture agents that are not hybridized to a probe) and their associated oligonucleotides still bound to the sample. In these embodiments, the labels may be removed by cleaving at least one bond in the probes that are associated with the sample, or a linker that links the probes to the labels, thereby releasing the labels from the probes. This cleavage can be done enzymatically, chemically or via exposure to light. Alternatively, the labels can be inactivated by photobleaching or by chemically altering the label).

If removal step is not done by removing the hybridized probes from the sample by denaturation, then a variety of chemical-based, enzyme-catalyzed or photo-induced cleavage methods may be used. For example, in some embodiments, the probes may contain a chemically or photo-cleavable linkage so that they can be fragmented by exposure to a chemical or light. In some embodiments, the duplexes (because they are double stranded) may be cleaved by a restriction enzyme or a double-stranded DNA specific endonuclease (a fragmentase), for example. In some embodiments, the probe may contain a uracil (which can be cleaved by USER), or may contain a hairpin that contains a mismatch, which can be cleaved using a mismatch-specific endonuclease. In some of these embodiments, after cleavage the Tm of the fragment of the probe that contains the label may be insufficiently high to remain base paired with the oligonucleotide and, as such, the fragment can disassociate from the oligonucleotide. In some embodiments, the probe and the label may be connected by a photo-cleavable or chemically-cleavable linker. Cleavage of this linker can release the label from the sample. In other embodiments, the probe may be an RNA, and the probe can be degraded using an RNAse. In some embodiments, an enzymatically cleavable linkage can be used. For example, esters can be cleaved by an esterase and a glycan can be cleaved by a glycase. Alternatively, the label itself may be inactivated by modifying the label. In one example, the dye may be photobleached, but other methods are known.

In some embodiments, after reading the sample, the method may comprise (e) removing the probes hybridized in step (c) from the sample by denaturation (i.e., by un-annealing the labeled probes from the oligonucleotides and washing them away), leaving the capture agents of (b) and their associated oligonucleotides still bound to the sample. This step may be done using any suitable chemical denaturant, e.g., formamide, DMSO, urea, or a chaotropic agent (e.g., guanidinium chloride or the like), using a toehold release strategy (see, e.g., Kennedy-Darling, Chembiochem. 2014 15: 2353-2356), or using heat, base, a topoisomerase or a single-strand binding agent (e.g., SSBP). This step can also be achieved through hybridization of an oligonucleotide with a greater affinity (e.g. PNA). In some cases, the probes may by removed by incubating the sample in 70% to 90% formamide (e.g., 75% to 85% formamide) for a period of at least 1 minute (e.g., 1 to 5 mins), followed by a wash. This denaturation step may be repeated, if necessary, so that all of the hybridized probes have been removed. As would be apparent, this step is not implemented enzymatically, i.e., does not use a nuclease such as a DNAse or a restriction enzyme, and does not result in cleavage of any covalent bonds, e.g., in any of the probes or oligonucleotides or removal of any of the capture agents from the sample. In this step, the strands of the probe/oligonucleotide duplexes are separated from one another (i.e., denatured), and the separated probes, which are now free in solution, are washed away, leaving the capture agents and their associated oligonucleotides intact and in place.

If a cleavable linkage is used (e.g., in the probes or to connect the probes to the labels, then the cleavable linker should be capable of being selectively cleaved using a stimulus (e.g., light or a change in its environment) without breakage of bonds in the oligonucleotides attached to the antibodies. In some embodiments, the cleavable linkage may be a disulfide bond, which can be readily broken using a reducing agent (e.g., □-mercaptoethanol or the like). Suitable cleavable bonds that may be employed include, but are not limited to, the following: base-cleavable sites such as esters, particularly succinates (cleavable by, for example, ammonia or trimethylamine), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable bonds can be apparent to those skilled in the art or are described in the pertinent literature and texts (e.g., Brown (1997) Contemporary Organic Synthesis 4(3); 216-237). A cleavable bond may be cleaved by an enzyme in some embodiments.

In particular embodiments, a photocleavable (“PC”) linker (e.g., a uv-cleavable linker) may be employed. Suitable photocleavable linkers for use may include ortho-nitrobenzyl-based linkers, phenacyl linkers, alkoxybenzoin linkers, chromium arene complex linkers, NpSSMpact linkers and pivaloylglycol linkers, as described in Guillier et al (Chem Rev. 2000 Jun. 14; 100(6):2091-158). Exemplary linking groups that may be employed in the subject methods may be described in Guillier et al, supra and Olejnik et al. (Methods in Enzymology 1998 291:135-154), and further described in U.S. Pat. No. 6,027,890; Olejnik et al. (Proc. Natl. Acad Sci, 92:7590-94); Ogata et al. (Anal. Chem. 2002 74:4702-4708); Bai et al. (Nucl. Acids Res. 2004 32:535-541); Zhao et al. (Anal. Chem. 2002 74:4259-4268); and Sanford et al. (Chem Mater. 1998 10:1510-20), and are purchasable from Ambergen (Boston, Mass.; NHS-PC-LC-Biotin), Link Technologies (Bellshill, Scotland), Fisher Scientific (Pittsburgh, Pa.) and Calbiochem-Novabiochem Corp. (La Jolla, Calif.).

### Iterative Methods

Methods herein can comprise steps that are repeated. In some cases, this can comprise repeating steps of the method. This can allow for a greater plurality of biological features of interest to be detected than can be accomplished without repeating the steps.

After removal of the probes, the sample may be hybridized with a different set of labeled probes which can bind to a different subset of amplified oligonucleotides (e.g., a second sub-set of two to four labeled probes, where the probes are distinguishably labeled), and the sample may be re-read to produce an image showing the binding pattern for each of the most recently hybridized sub-set of probes. In this manner, in different iterations of reading the sample, different biological features of interest can be detected. After the sample has been read, the probes may be removed from the sample, e.g., by denaturation or another method (as described above), and the hybridization and reading steps may be repeated with another different set of distinguishably labeled probes which can bind to another different subset of amplified oligonucleotides. In other words, the method may comprise repeating the hybridization, label removal or inactivation and reading steps multiple times with a different sub-set of two to four of the labeled nucleic acid probes, where the probes in each sub-set are distinguishably labeled and each repeat is followed by removal of the probes, e.g., by denaturation or another method (except for the final repeat) to produce a plurality of images of the sample, where each image corresponds to a sub-set of labeled nucleic acid probes. The hybridization/reading/label removal or inactivation steps can be repeated until all of the biological features of interest have been analyzed.

Nucleotide sequences used may be selected in order to minimize background staining, either from non-specific adsorption or through binding to endogenous genomic sequences (RNA or DNA). Likewise, the hybridization and washing buffers may be designed to minimize background staining either from non-specific adsorption or through binding to endogenous genomic sequences (RNA or DNA) or through binding to other reporter sequences.

In addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with haematoxylin and eosin (H&E).

### Kits

Also provided by this disclosure are kits that contain reagents for practicing the subject methods, as described above.

A kit can comprise two or more capture agents linked to oligonucleotides. A kit can be structured such that each different capture agent is linked to a different oligonucleotide. In some cases, a capture agent can be pre-linked to an oligonucleotide. In some cases, a capture agent can be packaged separately from an oligonucleotide, e.g., wherein the kit comprises instructions for linking the capture agents to the oligonucleotides. In some cases, a kit can comprise one or more reagents for linking the capture agent to the oligonucleotide.

A kit can comprise a circular nucleic acid primer. A kit can be structured such that each different oligonucleotide corresponds to a different circular nucleic acid primer. The circular nucleic acid primer can be joined, or pre-circularized. In some cases, the circular nucleic acid primer can be linear (e.g., a padlock probe). In such cases, a kit can comprise instructions for circularizing the nucleic acid primer, e.g., after binding to the oligonucleotide. In some such cases, a kit can comprise a reagent, such as a ligase, buffer, or other reagent, for ligating the circular nucleic acid primer.

A kit can comprise reagents for crosslinking the capture agents to a sample. In some cases, such a kit can comprise paraformaldehyde, formaldehyde, methanol, ethanol, acetone, a combination thereof, or another chemical capable of crosslinking the capture agents to the sample. In some cases, reagents can be pre-mixed, e.g., as a crosslinking buffer. In some cases, reagents can be separate, with instructions for mixing together (e.g., to make a crosslinking buffer). In some cases, for example if the reagents are commonly accessible, a kit may not provide the reagents, but can provide instructions for making a crosslinking buffer and using it to crosslink capture agents to the sample.

A kit can comprise reagents for carrying out a rolling circle amplification reaction. Such reagents can be, for example, BSA, a polymerase (e.g., Phi29 polymerase, or another polymerase such as those described herein), dNTPs, and/or reaction buffer appropriate for the polymerase. In some cases, the kit can additionally comprise instructions for carrying out the rolling circle amplification.

A kit can comprise probes, each comprising a label. A kit can be structured such that each different oligonucleotide can correspond to a different probe. The probe can be linked to the label, e.g., by a linker described herein. In some cases, a probe can be pre-linked to a label. In some cases, a probe can be packaged separately from a label, e.g., wherein the kit comprises instructions for linking the label to the probe.

A kit can comprise instructions for imaging a sample with the labeled probes bound. In some cases, such a kit can comprise instructions and/or reagents which allow multiplexing of the imaging protocol. For example, a kit can comprise instructions and/or reagents for chemically removing, inactivating, quenching, cleaving, or dehybridizing the probe or label.

In addition to above-mentioned components, a kit can further include instructions for using the components of the kit to practice the subject methods, i.e., instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment can be a kit that includes a web address where the instructions can be viewed and/or from which the instructions can be downloaded. As with the instructions, this means for obtaining the instructions is recorded on a suitable substrate.

### Computer Systems

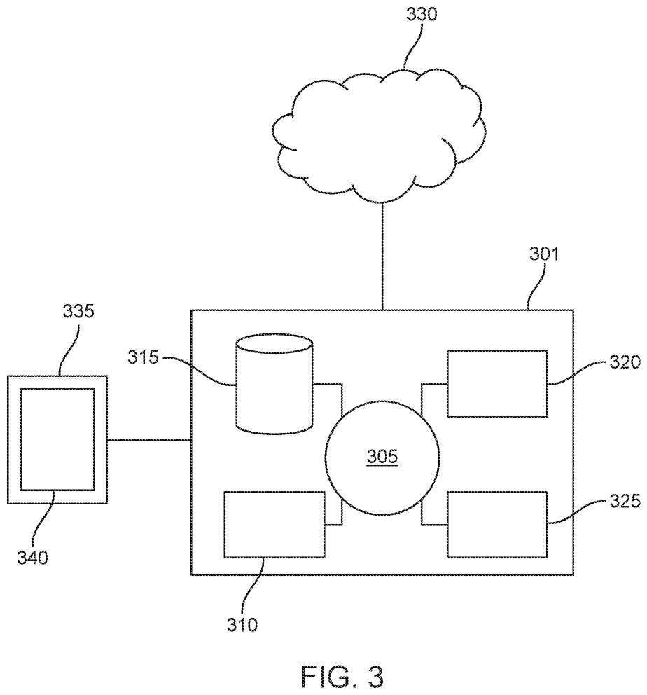

The present disclosure provides computer systems that are programmed to implement methods of the disclosure. FIG. 3 shows a computer system 301 that is programmed or otherwise configured to perform methods described herein. Computer system 301 can for example be configured to control delivery of 1) a solution comprising a plurality of oligonucleotides conjugated to capture agents as described herein, 2) wash buffer, 3) a fixing agent, 4) circular nucleic acid primers, and 5) reagents for executing an RCA reaction, or any subset of these components to a sample. The computer system 301 can regulate various aspects of methods of the present disclosure, such as, for example, amount of capture agent delivered, duration of capture agent incubation, length and timing of washes, amount of fixing agent delivered, duration of fixing agent incubation, amount of circular nucleic acid primer delivered, hybridization of a circular nucleic acid, a PCR amplification protocol including timing, temperature, etc. of cycles, amount of probe delivered, duration of probe incubation, and/or detection of probes including controlling imaging equipment and/or software. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 1135 that comprises a user interface (UI) 340 for providing, for example, instructions for carrying out methods described herein, user control of various steps of the methods, or analysis of images collected. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, control methods described herein, create oligonucleotide/circular nucleic acid primer/probe sets, or analyze images and/or data collected.

### EXAMPLES

### Example 1: Amplification on Fresh Frozen Tissue

Human fresh frozen tonsil tissue was applied to a slide and fixed with acetone. The tissue was hydrated and washed with buffer A (10 mM Tris &.5, 10 mM MgCl2, 150 mM NaCl, 0.1% Triton×100). The tissue sample was then fixed with 1.6% paraformaldehyde, and again washed with buffer A. The tissues were incubated with antibodies linked to oligonucleotides comprising barcodes (abbreviated BX), denoted CD45-BX001 (specific to CD45), CD4-BX002 (specific to CD4), and CD2-BX021 (specific to CD2) in buffer A for XX minutes for 3 hours. Each oligonucleotide comprised a barcode sequence as presented in Table 1:

TABLE 1
Barcode sequences

| Barcode ID | Barcode sequence | SEQ. ID. |
|---|---|---|
| BX001 | 5′-CTGATGGTTTAGGACTAC-3′ | 1 |
| BX002 | 5′-GATTGGTCCACTAACGTA-3′ | 2 |
| BX021 | 5′-CTATTATCATGAGGAGCG-3′ | 3 |

The samples were then washed with buffer A XX times for XX minutes each to remove excess antibody, and the antibodies were fixed onto the samples with 1.6% cold paraformaldehyde, cold methanol, and BS3. Slides were kept at 4° C. until the amplification step.

Next, the oligonucleotides were contacted with a padlock probe, which served as the circular nucleic acid probe. The padlock probe sequences are given in Table 2:

TABLE 2
Padlock probe sequences

| Barcode | | SEQ. |
|---|---|---|
| ID | Padlock probe sequence | ID. |
| BX001 | 5′- | 5 |
| | AACCATCAGTACTATCGTAACACATCCATAAGACCATCTGGCA | |
| | CGATTAGATGTAGTCCTA-3′ | |
| BX002 | 5′- | 6 |
| | GGACCAATCGACTCGGCGAGGAGTAGATACTATGGACTGTACA | |
| | CATGACTTACGTTAGT-3′ | |
| BX021 | 5′- | 7 |
| | TGATAATAGTCGATACGAGTGTATAACCCAGGTGATTCGACTT | |
| | GACGCAACGTTACGCTCCTCA-3′ | |

The amino acids at the 5′ end of the padlock probes were designed to be complimentary to the reverse complement of a portion of the barcode sequence, while the amino acids at the 3′ end of the padlock probes were designed to be complimentary to the complement of a portion of the 3′ end of the barcode sequence. In this way, when the padlock probes came into contact with the barcode sequences, base pair bonding occurred such that the padlock probe bound to the barcode sequence in a circular fashion.

The padlock probes were incubated on the samples in ligation buffer, such that the padlock probes bound to the oligonucleotides were ligated to become circular. The ligation buffer additionally comprised T4 DNA ligase (ligating enzyme) and salmon sperm DNA (for blocking). The recipe for the ligation buffer can be found in Table 3.

TABLE 3
Ligation buffer

| Ingredient | Amount | |
|---|---|---|
| T4 DNA ligation buffer | 1X | |
| Padlock probe | 250 | nM |
| T4 DNA ligase | 0.05 | U/μL |
| SSC buffer | 1X | |
| Salmon sperm DNA | 300 | ng/ml |

Samples were then washed in PBS to remove DNA ligase, probes, and salmon sperm DNA.

An RCA reaction was then carried out to amplify the oligonucleotides. The barcodes were incubated with Phi29 polymerase in amplification buffer for 1 hour at 37° C. The recipe for the amplification buffer can be found in Table 4.

TABLE 4
Amplification buffer

| Ingredient | Amount | |
|---|---|---|
| BSA | 1 | mg/ml |
| Phi29 polymerase | 1 | U/μl |
| dNTPs | 250 | μM |

| | |
|---|---|
| Phi29 polymerase buffer | 1X |

The samples were washed and incubated in 20% DMSO for 10 minutes, and then incubated with labeled probes for 5 minutes. The samples were then washed with 20% DMSO, followed by a wash with 1XAC1, and imaging using a fluorescent microscope. Sequences of labeled probes specific to barcodes used are provided in Table 5.

TABLE 5
Labeled probe sequences

| Barcode | Reporter | | SEQ. | |
|---|---|---|---|---|
| ID | ID | Labeled probe sequence | ID. | Fluorophore |
| BX001 | RX001 | 5′-TACGTTAGTGGACCA-3′ | 8 | Alexa750 |
| | | 5′-CGGCGAGGAGTA-3′ | 9 | Alexa750 |
| BX002 | RX002 | 5′-GTAGTCCTAAACCAT-3′ | 10 | ATTO550 |
| | | 5′-ATCGTAACACATCCA-3′ | 11 | Cy5 |
| BX021 | RX021 | 5′-CGCTCCTCATGATAA-3′ | 12 | Cy5 |
| | | 5′-ACGAGTGTATAACCC | 13 | ATTO550 |

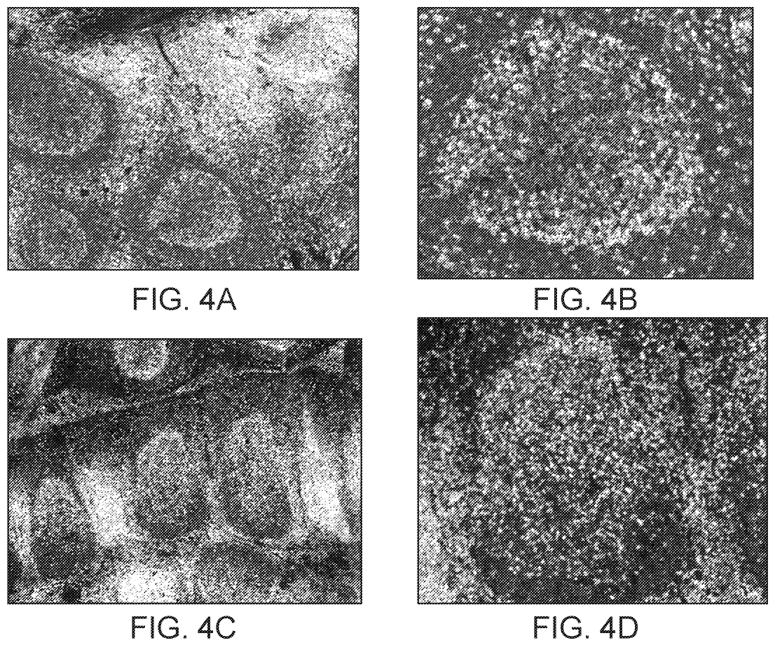

The images obtained of the probed human fresh frozen tonsil tissue are provided in FIG. 4. Panel A shows CD45-BX001 (Exposure time-50 ms) and CD4-BX021 (Exposure time 20 ms). Panel B shows a zoomed in portion of panel A. Panel C shows CD2-BX002 (Exposure time 20 ms). Panel D shows a zoomed in portion of panel C.

### Example 2: Oligonucleotide Design

Pipelines were used to create padlock probes for rolling circle amplification of oligonucleotides.

Random 42mer oligonucleotides were generated and filtered for GC content (36%≤GC %≤55%), Tₘ (66° C.≤Tₘ≤72° C.), tetramers, and homopolymers. Python and Biopython were used for the 42mer generation and filtering.

5mer, timer, 7mer, and 8mer oligonucleotide count tables of barcodes were generated using Perl. Sequences with high counts were filtered out using Kmer.

The barcodes were blasted against the 42mer oligonucleotides, and the 42mer oligonucleotides were blasted against the human genome, human transcripts, human rDNAs, the mouse genome, mouse transcripts, and mouse rDNAs using NCBI-Blast software.

At this point, there were 96 in-use barcodes. These 96 barcodes were split according to their thermodynamic mid-point to create “sticky end” sequences using Python. Table 6 shows sequences that were generated using this approach.

TABLE 6
Designed oligonucleotides

| | | Pad-lock | | | | | |
|---|---|---|---|---|---|---|---|
| | SEQ. | probe | SEQ. | Barcode | SEQ. | Reporter | |
| ID | ID. | sequence | ID. | sequences | ID. | sequences | Fluoro-phore |
| BX001/ | 14 | 5′- | 15 | 5′- | 16 | 5′- | Alexa750 |
| RX001 | | AACCAT | | CTGATG | | TACGTTAG | |
| | | CAG | | GTTTAG | | TGGACCA- | |
| | | AGTAAC | | GACTAC- | | 3′ | |
| | | CGGGTA | | 3′ | | | |
| | | TCTGCCG | | | | | |
| | | CATTATA | | | | | |
| | | TGAACG | | | | | |
| | | GTGTCTA | | | | | |
| | | GGGGTA | | | | | |
| | | GTCCTA- | | | | | |
| | | 3′ | | | | | |
| BX002/ | 17 | 5′- | 18 | 5′- | 19 | 5′- | ATTO550 |
| RX002 | | GGACCA | | GATTGG | | GTAGTCCT | |
| | | ATC | | TCCACT | | AAACCAT- | |
| | | TGAAATT | | AACGTA- | | 3′ | |
| | | CATTCGC | | 3′ | | | |
| | | AACTCTC | | | | | |
| | | GGCATA | | | | | |
| | | TCGACG | | | | | |
| | | GTACCTT | | | | | |
| | | TG | | | | | |
| | | TACGTTA | | | | | |
| | | GT-3′ | | | | | |
| BX003/ | 20 | 5′- | 21 | 5′- | 22 | 5′- | Cy5 |
| RX003 | | CATCCAC | | AGGTGG | | ATCGTAAC | |
| | | CT | | ATGTGT | | ACATCCA- | |
| | | CCAGAC | | TACGAT- | | 3′ | |
| | | TATATGG | | 3′ | | | |
| | | CCCGAC | | | | | |
| | | ACATTG | | | | | |
| | | GCGAAC | | | | | |
| | | TAGTTGT | | | | | |
| | | GCAC | | | | | |
| | | ATCGTA | | | | | |
| | | ACA-3′ | | | | | |
| BX004/ | 23 | 5′- | 24 | 5′- | 25 | 5′- | Alexa750 |
| RX004 | | CCTTATT | | AGAATA | | ACAATGA | |
| | | CT | | AGGGCT | | GCCCTTAT- | |
| | | TTAACTA | | CATTGT- | | 3′ | |
| | | AATCGC | | 3′ | | | |
| | | CGGAGA | | | | | |
| | | AAGCCT | | | | | |
| | | ATTGGA | | | | | |
| | | ATGTGCC | | | | | |
| | | CACG | | | | | |
| | | ACAATG | | | | | |
| | | AGC-3′ | | | | | |
| BX005/ | 26 | 5′- | 27 | 5′- | 28 | 5′- | ATTO550 |
| RX005 | | TAACCCT | | GAAGGG | | ACGAGTG | |
| | | TC | | TTATAC | | TATAACCC- | |
| | | ACACAT | | ACTCGT- | | 3′ | |
| | | ATCGATT | | 3′ | | | |
| | | TCTGGAC | | | | | |
| | | CGCAATT | | | | | |
| | | AATTGA | | | | | |
| | | CGTGTTG | | | | | |
| | | CC | | | | | |
| | | ACGAGT | | | | | |
| | | GTA-3′ | | | | | |
| BX006/ | 29 | 5′- | 30 | 5′- | 31 | 5′- | Cy5 |
| RX006 | | CCGCTTT | | ACAAAG | | ACCGTAA | |
| | | GT | | CGGTCT | | GACCGCTT- | |
| | | ACTACCC | | TACGGT- | | 3′ | |
| | | GGTATG | | 3′ | | | |
| | | CCAACCT | | | | | |
| | | TAATCG | | | | | |
| | | AGGTAA | | | | | |
| | | CGATCCT | | | | | |
| | | GCA | | | | | |
| | | ACCGTA | | | | | |
| | | AGA-3′ | | | | | |

### Example 3: Rolling Circle Amplification on FFPE Tissue

Human FFPE tonsil tissue was prepared on slides and dewaxed and subjected to a standard antigen retrieval protocol in Tris/EDTA buffer in preparation for staining.

The samples were then incubated with antibodies directed toward CD31 (labeled CX001) and CD3 (labeled CX002), wherein the antibodies were linked to oligonucleotides. The sample/antibody/oligonucleotide complex was incubated with padlock probes corresponding to the oligonucleotides for 2 hours at 37° C. The ligation buffer for this step is the same as that described in Table 3.

CX001 and CX002 were incubated with corresponding padlock probes for ligation at 37° C. for 30 minutes. The padlock probes were designed such that after the rolling circle amplification step, the labeled probe binds to the same sequence as the padlock probe.

Antibodies ligated to padlock probes were then incubated on the tissue for 2 hours. The buffer for this step is the same as described in Table 2.

The tissue samples were washed with PBS, and rolling circle amplification was executed. incubated with Phi29 polymerase in amplification buffer for 1 hour at 37° C. The recipe for the amplification buffer can be found in Table 4.

The tissue samples were washed in PBS and incubated with 1.6% paraformaldehyde, Cold MeOH and BS3 to crosslink the antibodies to the tissue, followed by another wash in PBS. Tissue samples were stored at 4° C. until imaging.

Tissue samples were washed and incubated in 20% DMSO for 10 minutes. Then, the tissue samples were incubated the labeled probes for 5 minutes, followed by a wash with 20% DMSO, a wash with 1XAC1, and imaging using a fluorescent microscope. Reporters used for hybridization were AlexaFluor-750-RX001, Atto550-RX002.

A clear tissue protocol was used to de-hybridize the labeled probes for removal, and the tissue samples were again imaged using a fluorescent microscope to verify removal of the labeled probes.

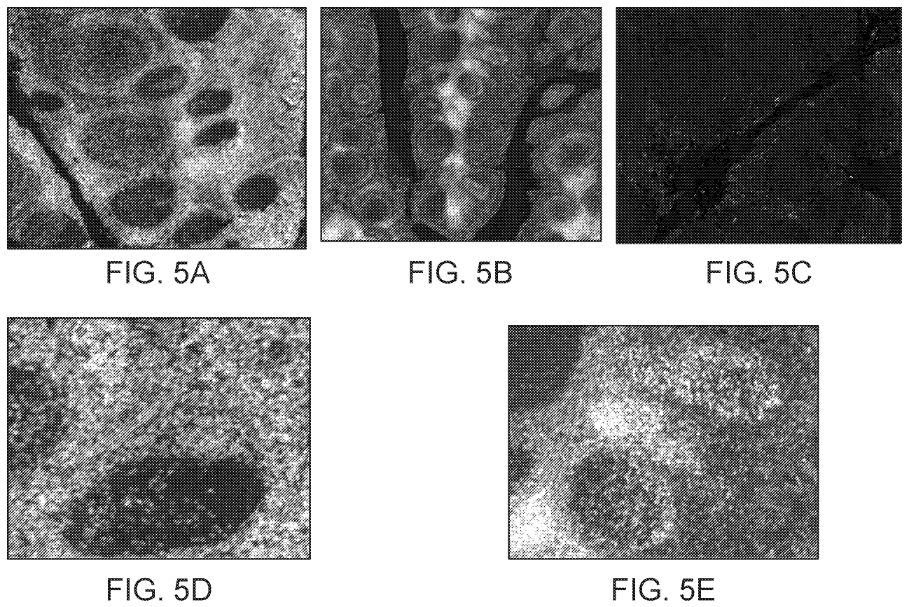

Images of the human FFPE tonsil tissue stained with antibodies linked to oligonucleotides from this experiment are shown in FIG. 5. Panels A and B show CD31-CX001 (exposure time-50 ms) and CD3-CX002 (exposure time 20 ms) and. Panel C depicts the tissue sample after removal of the labeled probes via de-hybridization. Panels D and E present zoomed-in regions of panels A and B, respectively.

### Example 4: An RCA-Based Amplification Strategy

Oligonucleotide-tagged antibodies (e.g., CODEX antibodies) can be employed in an RCA based strategy to amplify the antibody signal by 20× as measured through dye-tagged oligonucleotide hybridization relative to the standard CODEX technology for all three fluorescence channels used (FAM, Cy3 and Cy5).

Isothermal amplification of cDNA using Phi29 polymerase can be an efficient method of creating copies of a repeated oligonucleotide sequence. A repeated sequence can increase the signal associated with each CODEX-tagged antibody by creating multiple binding sites for CODEX-tagged dyes per antibody conjugation site. Antibodies such as, for example, anti-human CD3 (clone: UCHT1), anti-human CD19 (clone: HIB19) and anti-human CD31 (clone: WM59) can be used to stain fresh-frozen human tonsil tissues using the standard CODEX workflow. Antibody staining can be visualized using a Keyence BZ-X700 inverted microscope. In some cases, quantitation can be assessed using ImageJ, and can involve the use of segmentation-based algorithms. Staining data from non-amplified hybridization experiments (i.e. the current CODEX workflow) can be compared to data generated from RCA-based samples using the integrated signal intensity values, which can be generated from segmentation analyses.

Optimization of RCA conditions for CODEX detection: The current (non-RCA) CODEX workflow can comprise: 1) antibody conjugation and panel development, 2) tissue staining with CODEX-tagged antibodies and 3) CODEX image acquisition and fluidic cycling. Amplification of antibody-conjugated DNA sequences can be efficient and have the least experimental complexity when performed after antibody staining and tissue fixation and prior to CODEX data acquisition. At this step, all bound CODEX-tagged antibodies can be adhered to the tissue specimen through either or both paraformaldehyde and NETS-ester based cross-linking. Manipulation of samples to perform the RCA step can thus be performed on the tissue surface and not in solution.

The parameter space associated with RCA methodologies can include: cDNA and primer thermodynamic properties, polymerase enzyme:substrate ratios, nucleotide concentrations, buffer composition, salt concentration and incubation time and temperature. As a starting place, conditions related to enzyme and buffer conditions previously used to amplify DNA conjugated to an antibody moiety can be emulated while the oligonucleotide sequence space is optimized. There may be three components to the cDNA structure: 1) the binding site (a′) to the antibody-conjugated oligonucleotide (a), 2) the binding site (b′) to the CODEX-tagged dye (b) and 3) the sequences between these two binding sites. The oligonucleotide properties for the CODEX-tagged dye binding region were carefully worked out for the development of the existing CODEX platform. For these sequences, it can be important to have sufficient length and associated thermodynamic properties to ensure binding at room temperature using the optimized CODEX hybridization buffers and complete removal in the presence of the CODEX removal buffers. A set of these sequences can be used as sequences b and b′ for the design of the RCA compatible oligonucleotides for screening purposes. For the design of sequences a and a′, different sequence lengths with correspondingly different thermodynamic properties can be screened. Optimal sequence properties for these DNA components should result in efficient and specific ligation and effective amplification using phi29 polymerase. Sequences that are too short can have inefficient or transient binding to the associated CODEX antibody tags and can therefore result in lower or no signal enhancement.

Ligation can be carried out based on previously described conditions. Reproducible ligation efficiency, as measured by gel electrophoresis, of greater than 90% can be deemed successful. Results from RCA-based CODEX screening experiments using the three antibody clones listed above can be quantified and compared to results obtained from a standard CODEX workflow. Equivalent exposure times and other associated microscope settings can be used to enable direct comparisons of signal sensitivity. A variety of conditions related to buffer composition, enzyme:substrate ratios and incubation times can be screened to determine the optimal enhancement in signal intensity. An increase in signal intensity of at least 20× can result. Screening optimized conditions with antibodies against low-level markers: The three antibody clones used to develop the RCA compatible CODEX conditions target abundant cell surface markers within human tonsil tissue. These antibodies work well as positive controls to ensure the methodology is working. utility of this signal enhancement can be for the measurement of less abundant markers. To this end, these optimized conditions can be applied towards the detection of a variety of markers that have previously proved elusive using the standard CODEX technology. These markers include: GATA-3, T-bet, CD25, pSTAT1 and RORγT. Purified antibodies for each of these markers can be conjugated to RCA compatible CODEX sequences and can be used to stain fresh-frozen human tonsil, lymph node and melanoma tissues. It is possible that some of these antibodies can still fail to reveal relevant marker staining. This can be due to a variety of reasons, including a lack of expression within the test tissues, loss of antigen binding due to antibody conjugation, loss of antigen binding due to tissue subjection to acetone (part of the CODEX staining protocol) or a lack of effective signal enhancement. Control experiments using the same clones and tissue samples can be performed using a standard IF workflow with detection by polymer enhanced secondary antibodies as a means of eliminating some of these explanations. These experiments can be deemed successful if three of the five markers become detectable by CODEX with associated signal to noise ratios of at least 5.

### Example 5: Validation of RCA-Enhanced Methods

Multiplexed RCA-enhanced methods (e.g., CODEX) can be performed using tagged antibodies and associated oligonucleotide reagents to demonstrate the feasibility of developing a full panel (e.g., 40+ markers) using iterative cycles of adding and removing of dye tags. Each antibody channel can be amplified by a factor of 20× relative to the standard platform, and there may be no overlap in signal between samples, as measured by fluorescence signal and known staining patterns of associated markers.

The CODEX technology is based upon a library of sequence orthogonal probes and controlling the addition and removal of complementary dye-labeled tags to reveal subsets of a large panel of tagged antibodies per cycle. Each probe pair may not interfere with the signal of other sequences and does not bind endogenous DNA or RNA components. Additionally, the library of CODEX probe pairs can have similar thermodynamic properties such that binding and removal of complementary dye tags can be accomplished with the same set of buffers. To implement CODEX signal enhancement with RCA for a library of CODEX-tagged antibodies one or more of three criteria can be present: 1) design of sequence orthogonal oligonucleotide sets, 2) optimization of efficient and reproducible conditions to promote and dissociate hybridization pairs and 3) protection of oligonucleotides bound to antibodies that can not be subjected to the amplification process The present disclosure describes using RCA enhancement to a standard CODEX antibody panel, which are generally between 30-40 markers. This can be accomplished through development of conditions to meet each of the aforementioned criteria on a smaller panel (e.g., six markers). A larger developmental effort to create a full library of RCA CODEX compatible probe sets can be undertaken during the Phase II portion of this grant application.

Each RCA oligonucleotide set can comprise of two regions: the binding region to the antibody oligonucleotide tag (a and a′) and the binding region to the CODEX-tagged dye (b and b′). The properties required for these latter sequences (b and b′) should be equivalent to those used in the current implementation of the CODEX platform. Optimal sequence length and corresponding melting temperatures can be determined for a and a′. Ten sets of RCA compatible CODEX sequences can be designed using a publicly available random DNA sequence generator (https://www.random.org/sequences/) based on the thermodynamic property requirements for a and b (and by extension a′ and b′). Sequence overlap between the different RCA compatible CODEX tag sets and overlap with endogenous human RNA and DNA components can be screened using NCBI PrimerBLAST (www.ncbi.nlm.nih.gov/tools/primer-blast/). The resultant group of probe sets can be used to generate a six-marker antibody panel comprised of the three antibodies targeting abundant markers used in the screening and optimization experiments (CD3, CD19 and CD31) in addition to the three validated low-level markers from the previous specific aim. Each CODEX-tagged antibody can be screened individually to optimize the antibody staining conditions and associated exposure times. A two cycle RCA-enhanced CODEX experiment can be carried out with these six markers revealing half of them on the first cycle using the three standard fluorescence channels (FAM, Cy3 and Cy5) and half on the second cycle using the same fluorescence dyes. Standard hybridization and probe removal conditions based on the current implementation of the CODEX platform can be tested to determine whether CODEX-tagged dyes can be effectively removed. This can be measured by imaging the staining pattern after CODEX-tag dye hybridization and after removal. The fluorescence signal where positive staining was measured can be completely removed and equivalent to background signal for successful removal. It is possible that these conditions can be optimized to accommodate the difference in DNA structure after RCA compared with the short oligonucleotide sequence used in the absence of amplification. A variety of parameters can be screened, if necessary, to optimize the removal of bound CODEX-tagged dyes including: DMSO concentration, salt concentration and composition and removal buffer incubation time. The thermodynamic properties of b and b′ can be altered to promote effective CODEX-tag dye removal. This can be tested if altering the buffer composition does not result in effective CODEX-tag dye removal. Success can be measured against the staining pattern and signal achieved for each antibody individually, where signals obtained during the CODEX experiment within 10% deviation of these values can be deemed acceptable.

For some implementations of the RCA-enhanced CODEX experiments, it may be useful to amplify some antibody signals but not others. This could be the case when simultaneously measuring markers with a large difference in levels of abundance and/or antibody binding effectiveness. In these cases, it may not be necessary to amplify the signal of the very high-level markers, but can be necessary to increase the signal for the very low-level markers. Phi29 polymerase has both 5′-*3′ polymerization activity and a 3′-*5′ exonuclease activity. To incorporate the RCA step into the CODEX workflow, it can be necessary to ensure oligonucleotides bound to antibody moieties not destined for amplification are protected from this potential exonuclease degradation. Sequences used to tag antibodies can be modified at or near the 3′ end with covalent groups known to inhibit this nuclease activity, including phosphorothioate bonds, 2′-O-Methyl-, inverted dT, phosphorylation and PEG spacers25. Exonculeoase activity can be measured using a standard CODEX staining experiment, where the oligonucleotide bound to tissue bound antibodies would in theory be degraded upon addition of phi29 polymerase. The optimal modification can be selected based on prevention of oligonucleotide degradation and synthesis cost considerations. These optimized sequences can be incorporated into the same two-cycle CODEX experiment described above, with CD3, CD19 and CD31 containing modifications to prevent degradation by phi29 polymerase. Fluorescence intensity for these markers can be compared to samples stained in the absence of phi29 and signal intensities within 10% deviation of these values can validate sequence fidelity during the RCA process.

The signal enhancement strategy is extended for the CODEX platform for use with a full antibody panel and for the measurement of a variety of marker types. As part of this effort, a library of more than 50 RCA compatible CODEX sequences can be designed and validated. Correspondingly, multiple antibody clones against transcription factors, cell signaling proteins and other potentially low-level markers which are currently below the signal threshold using the existing CODEX platform can be screened for use with the RCA-enhanced CODEX methodology. Additionally, enhanced software analysis tools can be developed to enable extraction of meaningful biological data from CODEX data across a variety of marker types. Finally, the reagents and associated analysis software for RCA-enhanced CODEX can be deployed at two test sites for integrated use with potential and existing customers to confirm the utility of the integrated system and provide feedback for the production and manufacturing of these pieces.

### Example 6: Design and Screening of Oligonucleotide Sequence Library

A library of RCA-enhanced CODEX compatible oligonucleotide sequences can be designed and screened for use in a highly multiplex assay (e.g., greater than 50 parameters). Each oligonucleotide set can be sequence orthogonal and have similar amplification properties. In some cases, sequences can result in at least a 20× signal enhancement relative to the standard CODEX platform. Antibodies against intracellular targets can be screened for compatibility with this platform. Such design can result in some cases in at least 20 validated clones.

First, an RCA-enhanced CODEX oligonucleotide library can be generated. The present disclosure provides for pre-conjugated CODEX-tagged antibodies with associated RCA oligonucleotide components so users can select off-the-shelf antibody panels. For example, a library of 50 or more compatible RCA enabled CODEX probe sets can be used. Additionally, sequences can be provided in a conjugation kit format such that users can create their own RCA-enhanced CODEX antibody panels for more tailored applications. A library size of 50 compatible oligonucleotide pair sets can be sufficiently large to enable both routes and produce a variety of pre-conjugated CODEX antibody products.

In an illustrative embodiment, the properties of an RCA-enhanced CODEX oligonucleotide library can be as follows: 1) oligonucleotide antibody bound (a) and detection sequences (b) contain sequence that are orthogonal such that there is no signal overlap between channels, 2) the thermodynamic properties for each of these sequences can be similar such that ligation and polymerization are efficient and reproducible for each set and the hybridization and removal of dye probes is accomplished with the same buffer conditions and 3) each oligonucleotide set, including the detection and amplification sequences, do not bind to endogenous DNA and RNA components across relevant species, including human and mouse. The design and generation of a small number of these oligonucleotide sets can be accomplished manually; however, for generating a library of this size, a custom software package can be developed that screens for these criteria in silico and creates a set of output sequences amenable to further screening. An algorithm or software package can generate candidate oligonucleotide sequences with the necessary thermodynamic properties, screen these sequences for overlap both within the candidate population and against mouse and human transcriptomes and genomes, and can eliminate CODEX-tagged dye sequences with known fluorescence interference. The resultant candidate sequences can be synthesized and screened for effective activity in an RCA-enhanced CODEX experiment.

The screening and validation process of candidate oligonucleotide sets can involve multiple steps. First, the ligation efficiency and polymerization rate can be measured in isolation for each set. To do this, each oligonucleotide set can be conjugated to anti-human CD3 antibody (clone: UCHT1) and used to stain human fresh-frozen tonsil tissue. The ligation efficiency can be determined based on gel electrophoresis, while the polymerization rate can be measured relative to a standard CODEX experiment, which can have direct hybridization to the non-amplified sequence. Ligation efficiencies of greater than 90% or polymerization rates resulting in at least 20-fold signal enhancement can be deemed successful. Next, sequences can be screened for signal overlap in relation to amplification sequences, detection sequences, or both. Samples of anti-mouse CD45 antibody can be conjugated to corresponding components from each oligonucleotide set and used to stain a sample of spleen cells, which can be isolated from wild-type mice. Each sample can be combined and spread onto a coverslip for further processing. Ligation, amplification and detection through hybridization can be carried out, for example by using the CODEX instrument. Each sample can be subjected to iterative addition and removal of associated CODEX-tagged dyes. Sequences that show signal overlap can be attributed to the amplification sequences, detection sequences, or both. Signal overlap can be problematic and may be eliminated. Based on this analysis, the minimal set of overlapping sequences can be removed from the candidate library. Finally, the remaining sequences can be screened for potential sequence overlap, which can result from association during the antibody staining step, where all antibodies can be stained in a single step. Each antibody can be conjugated to a unique, previously validated, antibody clone and can be used to stain multiple test tissues, including human tonsil, lymph node, and melanoma. Sequence overlap can be determined based on comparison of known staining patterns which can be shown for each antibody clone when stained independently to the staining that can result from an RCA-enhanced CODEX experiment.

A library of at least 50 RCA-enhanced CODEX compatible oligonucleotide sets can be generated. For smaller libraries, including libraries with significantly fewer than 50 oligonucleotide sets, a similar effort can be undertaken on a set of newly designed sequences. The characteristics of oligonucleotides resulting in failure due to each of these screening efforts can be evaluated, and the initial oligonucleotide design algorithm can be updated as needed to prevent similar failures in other cycles of sequence generation.

Antibody clones can be validated for the RCA-enhanced CODEX platform. Over 200 antibodies, which can be anti-mouse and anti-human antibody clones, can be validated for use with the CODEX technology on fresh-frozen tissues, and in some cases mostly target abundant cell surface markers. RCA-enhanced CODEX can provide increased sensitivity, and this increased sensitivity can enable detection of a variety of different marker types. To this end, antibody clones that target markers of lower expression. can be screened and validated. In some cases, 30-40 additional antibody clones can be screened and validated that target markers of lower expression. The standard antibody screening process can involve staining a variety of test tissues with the candidate CODEX-tagged antibody, in some cases in the presence of a directly dye-conjugated counterstain. The resultant signal pattern can be compared to the expected staining pattern of the counterstain as well as to publicly available databases (e.g. www.proteinatlas.org/tissue). Reasons for failure to see antibody signal during these screening experiments can include loss of antibody signal due to conjugation, insufficient expression in the test tissues, or both.

In some embodiments, if one or more antibody clones is not compatible with an antibody conjugation, alternative clones targeting the same antigen can be screened. For some markers, expression may be limited in multiple tissue types. To confirm expression in these instances, standard IHC assays using a purified primary antibody and polymer conjugated secondary antibody can be used. In some cases, up to about 20 antibody clones or more compatible with RCA-enhanced CODEX detection for fresh-frozen tissues can be validated.

Some or all validated antibody panels for the current implementation of the CODEX platform can be compatible with analysis of fresh-frozen tissues, formalin-fixed paraffin-embedded (FFPE) tissues, or both.

### Example 7: Software Analysis Modules

Software analysis modules for processing high-parameter RCA-enhanced CODEX datasets can contain staining data against intracellular markers.

The utility of the CODEX platform can be rooted in the data and associated analysis tools. For the first time, tissue samples can be analyzed not just with respect to the marker expression but additionally in relation to the associated spatial organization for up to about 40 or more targets. For the current implementation of the CODEX platform measuring abundant cell surface markers and extracting spatial associations between annotated cell types can be useful. Technical advancements can enable detection of additional markers by CODEX potentially expanding the types of targets that can be measured. An updated analysis pipeline and associated software package can enable extraction of meaningful biological data from these datasets.

Advanced segmentation-based analysis tools can improve analysis. Tools and methodologies for analyzing RCA enabled CODEX data using antibody panels targeting transcription factors, signaling molecules and diffuse extracellular proteins can be utilized. For transcription factors, signaling molecules, or both, the intracellular localization can be a critical parameter with potential biological implications. It can be important to differentiate between expression of these markers within the nucleus and within the cytoplasm. A subcellular segmentation algorithm can be used map each of these regions and associate the corresponding fluorescence signal with this location. The nucleus can be identified using Hoechst dye. The cytoplasm can be identified through subtraction of the nucleus relative to the signal from a cell surface marker. An additional relevant parameter can be the appearance of fluorescence signal, and optionally whether it is punctate or diffuse in nature. This parameter can be measured through analysis of the local fluorescence signal and fitting these patches to different models representative of either diffuse or punctate signal. Either or both of the subcellular localization and associated nature of the fluorescence signal can be provided in a data output file, which can contain the associated expression data for every cell within the tissue sample, optionally based upon the current segmentation algorithm or the corresponding spatial dimensions within the tissue or both. These cellular features can be used to cluster the cells using one or more of a variety of existing algorithms and tools, and in some cases the resultant cell types can be annotated. The addition of subcellular localization indicator tools can classify cells based on the cell surface marker expression, on their cellular state, or both. In some cases, classification can reveal important insights into disease mechanism and therapeutic modes of action.

Machine learning algorithms can be used for RCA-enhanced CODEX data analysis. Segmentation of tissue data can provide insight into the single-cell expression profile within this space. Compensation algorithms can mitigate limitations of this approach due to proximity of neighboring cell edges in some cases. In some cases, another segmentation approach, using use a combination of classic image analysis and machine learning, can identify and refine cell patches. An analysis tool can use these methods to analyze RCA-enhanced CODEX data. Patches of fluorescence signal across the parameter space of an RCA-enhanced CODEX dataset can be analyzed and used to train a machine learning model to map different combinations of signals, corresponding to different marker distributions. In some cases, an open source machine learning platform can be used to accomplish the analysis. This step can be followed by a fluorescence compensation step. The composition of patches identified in this analysis can consist of single cell data or data from neighboring cells or both. The cell borders may or may not be critical. In some cases, the signature of signal intensity for marker combinations becomes the unit of comparison. For example, patches identified through the machine learning algorithm consisting of CD4, CD19, pSTAT1 and Ki67 signal intensities can correspond to a CD4-helper T cell residing next to a B cell with signs of proliferation. In some cases, discrete differentiation between the signal associated with each individual cell may not be possible. In some cases, spatially driven associations between marker combinations can be identified. In some cases, the machine learning model can be trained on a subset of their data and then apply the resultant model to extract data across the remainder of their datasets. In some cases, this is easy for the user.

### Example 8: Integration of RCA Platform

The RCA-enhanced CODEX platform can be integrated, including biochemical reagents and associated analysis tools. This platform can be deployed to test sites to test compatibility of all components, obtain, feedback, or both. A test site can obtain and analyze data from an RCA-enhanced CODEX experiment using oligonucleotide-tagged antibodies that are pre-validated, developed at the test site, or both.

In some cases, increased sensitivity of the CODEX platform can lead to the validation of additional markers for use with this technology. In some cases, increased sensitivity of the CODEX platform can broaden the potential applications that can benefit from this type of analysis. Sets of reagents and associated software packages, or kits, can enable use of RCA-enhanced CODEX. In some cases, the kits can be used in combination with an instrument to analyze a multitude of different sample types. The reagent products can include pre-conjugated CODEX antibodies to RCA compatible oligonucleotides, oligonucleotide sequences that are amenable to conjugation by the end user for development of custom CODEX antibody panels, or both. Corresponding buffer can be used to perform both the RCA steps as well as the standard CODEX workflow. RCA-enhanced CODEX data acquisition can take place a CODEX instrument, which can be coupled a microscope infrastructure, optionally in a laboratory. Analysis of the resultant RCA-enhanced CODEX data can be performed using a toolkit.

Test sites can be equipped with a CODEX instrument and can receive a panel of CODEX-tagged antibodies. The panel can include about 10, 20, 30, 40, or more CODEX-tagged antibodies. The panel can include corresponding detection CODEX-tagged dyes, which can be optimized for use with mouse tissue applications, human tissue applications, or both. In some cases, the panel can include conjugation materials which can be used to create customized CODEX antibody panels. Buffers, associated reagents, or both can be provided based on the anticipated formulation of the CODEX antibodies. A field application scientist can be available to train the personnel on the relevant RCA-enhanced CODEX protocols. Each test site can collect at least 1, 5, 10, 20, 30, 40, or more datasets from one or more tissue types. Datasets can be collected using the RCA-enhanced CODEX platform. Datasets can be collected using the pre-conjugated CODEX-tagged antibody panel A custom set of CODEX-tagged antibodies for use with the RCA-enhanced CODEX platform can be designed and created. These antibodies can be added to the panel of pre-conjugated antibodies. Additional datasets can be collected. In some cases, success can be measured based on the comparison of data generated independently using the same tissue specimens. In some cases, different microscopes can be used to collect the data. In some cases, direct comparison of fluorescence signal intensity may be appropriate. In some cases, comparison of fluorescence signal intensity may not be the best metric. In some cases, comparison of fluorescence signal intensity may not be appropriate. In some cases, signal to noise ratios can be compared. In some cases, signal to noise ratio values of independent datasets within a threshold of each other can be considered successful. In some cases, signal to noise ratio values of independent datasets within a threshold of a gold standard value can be considered successful. Threshold values can be about 1, 5, 10, 15, 20, 25, or 30%.

The specificity of the antibody staining can be compared between samples to detect co-localization, to detect mutual exclusivity of different markers, optionally based on their associated biological function, or both.

### Example 9: Analysis of Human Tonsil Tissue

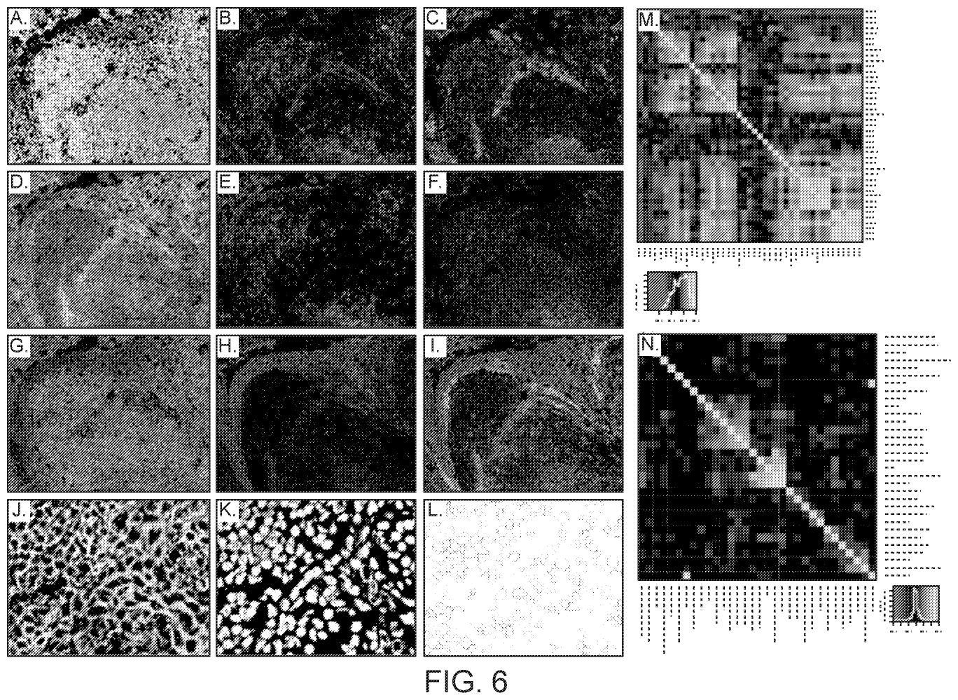

A fresh-frozen human tonsil tissue sample was stained with a 24-marker antibody panel as shown in FIG. 6. A) The nuclear stain was collected during each cycle and used for drift compensation. Images B-I show CODEX data collected across eight cycles and three fluorescent channels. Signals are listed in the order red, green, and blue, and correspond to data collected on the FAM, Cy3 and Cy5 channels, respectively. B) Collagen IV, CD7, Ki67, C) CD38, CD31, CD4, D) CD45, CD90, CD19, E) CD15, CD3, CD56, F) CD21, CD34, CD278, G) HLA-DR, CD22, CD279, H) CD8, CD57, Pan-cytokeratin, I) CD9, podoplanin, CD11c. An example of a segmentation pipeline is shown in panels J-L, where J) shows the zoomed-in overlay of data from a single CODEX cycle, K) shows the corresponding nuclear stain and L) shows the result of a segmentation algorithm for identifying individual cells. Fluorescence data for each segmented cell across all channels and cycles was integrated and listed in an output table with the associated spatial dimensions. This data can then be clustered using a variety of existing tools and annotated based on known cell types. Various downstream analyses can then be performed. M) An example heatmap showing the marker to marker spatial correlation from a tonsil tissue sample stained with 42 CODEX-tagged antibodies is shown. N) An example heatmap correlation analysis of annotated cell types within follicle regions of a tonsil tissue is shown.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for biological feature detection comprising:
contacting a sample comprising a plurality of biological features of interest with a plurality of capture agents, wherein each capture agent is capable of binding to a different biological feature of interest, wherein each capture agent is conjugated to a different oligonucleotide;
fixing the capture agents bound to biological features of interest to the sample;
contacting each oligonucleotide with a circular nucleic acid primer, wherein a segment of the nucleic acid primer is complimentary to the oligonucleotide, and wherein each oligonucleotide is contacted with a different nucleic acid primer;
amplifying the oligonucleotides using the circular nucleic acid primers as a template to yield amplified oligonucleotides;
contacting each of a subset of the oligonucleotides with a probe comprising a label to form a probe-amplified oligonucleotide duplex, wherein each probe can bind to only one oligonucleotide;
reading the sample to determine the binding pattern for each of the probes, inactivating or removing the labels, and repeating the contacting and reading steps with different probes that bind to a different subset of oligonucleotides.

2. The method of claim 1, wherein the sample is a biological sample.

3. The method of claim 1, wherein the sample is selected from the group consisting of a fresh sample, a frozen sample, and a chemically fixed sample.

4. The method of claim 1, wherein the sample is a FFPE tissue sample.

5. The method of claim 1, wherein the sample comprises a cell.

6. The method of claim 1, wherein the sample is selected from the group consisting of a biological tissue, a biological fluid, and a homogenate.

7. The method of claim 1, wherein the sample comprises cells.

8. The method of claim 7, wherein the cells comprise a rare cell population.

9. The method of claim 7, wherein the cells comprise cancer cells.

10. The method of claim 7, wherein the cell is selected from the group consisting of an animal cell, a plant cell, a bacterium, a fungal cell, or a protist.

11. The method of claim 1, wherein the sample a human sample or a mouse sample.

12. The method of claim 1, wherein the sample comprises a pathogen.

13. The method of claim 12, wherein the pathogen is selected from the group consisting of a bacterial cell, a yeast cell, a bacterial cell, a virus, a viral vector, or a prion.

14. The method of claim 1, wherein the sample comprises a tumor tissue.

15. The method of claim 1, wherein the sample comprises healthy tissue.

16. The method of claim 1, wherein the sample is adhered to a slide.

17. The method of claim 1, wherein the biological features comprise proteins.

18. The method of claim 1, wherein the biological features comprise markers.

19. The method of claim 18, wherein at least one of the markers is a low level marker.

20. The method of claim 18, wherein the biological features comprise a disease marker.

21. The method of claim 18, wherein the biological features comprise a diagnostic marker.

22. The method of claim 18, wherein the markers comprise a molecule selected from the group consisting of a transcription factor, a signaling molecule, a diffuse extracellular marker, or a cell surface marker.

23. The method of claim 1, wherein the biological features comprise a mutated protein.

24. The method of claim 1, wherein the capture agents comprise an antibody.

25. The method of claim 1, wherein the capture agents comprise an antibody fragment.

26. The method of claim 25, wherein the antibody fragment is selected from the group consisting of an IgG, an IgM, a polyclonal antibody, a monoclonal antibody, a scFv, a nanobody, a Fab, or a diabody

27. The method of claim 1, wherein each different oligonucleotide is at least 10 nucleotides long.

28. The method of claim 1, wherein each different oligonucleotide is at least 25 nucleotides long.

29. The method of claim 1, wherein each different oligonucleotide is no more than 100 nucleotides long.

30. The method of claim 1, wherein the fixing comprises crosslinking.

31. The method of claim 30, wherein the crosslinking comprises using formaldehyde.

32. The method of claim 1, wherein the circular nucleic acid primer is between 6 nucleotides long and 100 nucleotides long.

33. The method of claim 1, wherein the segment of the nucleic acid primer that is complimentary to the oligonucleotide is between 16 nucleotides long and 18 nucleotides long.

34. The method of claim 1, wherein the amplifying is performed using a polymerase.

35. The method of claim 1, wherein the polymerase is Phi29 polymerase.

36. The method of claim 1, wherein the amplifying step lasts for about 1 hour.

37. The method of claim 1, wherein the amplifying step is performed at about 37° C.

38. The method of claim 1, wherein each probe comprises a different label than each other probe.

39. The method of claim 1, wherein the probe-amplified oligonucleotide duplex can have a Tₘ of at least 15° C.

40. The method of claim 1, wherein the label can be a fluorescent label.

41. The method of claim 40, wherein the fluorescent label can be selected from the group consisting of Cy3, Cy5, Alexafluor555, Alexafluor647, Alexafluor750, POPO-3, TOTO-3, POPRO3, and TOPRO3.

42. The method of claim 1, wherein the fluorescent label can be attached to the probe by a linker.

43. The method of claim 1, wherein reading the sample comprises fluorescent imaging.
